SEQUENCE LISTING (18 sequences)

SEQ ID NO: 1 - protein, 114 aa, Homo sapiens
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLKKVIQKILDSGNKKN

SEQ ID NO: 2 - DNA, 30 bp, synthetic construct
  source 1..30: mol_type = other DNA
tgacggggtcacccacactgtgcccatcta

SEQ ID NO: 3 - DNA, 30 bp, synthetic construct
  source 1..30: mol_type = other DNA
ctagaagcatttgcggtggacgatggaggg

SEQ ID NO: 4 - DNA, 21 bp, synthetic construct
  source 1..21: mol_type = other DNA
gttgtcatcagcaccagcatc

SEQ ID NO: 5 - DNA, 20 bp, synthetic construct
  source 1..20: mol_type = other DNA
accacacagtcctctccagc

SEQ ID NO: 6 - DNA, 20 bp, synthetic construct
  source 1..20: mol_type = other DNA
cggacataccgagggacatg

SEQ ID NO: 7 - DNA, 21 bp, synthetic construct
  source 1..21: mol_type = other DNA
ccaacccacgaatgcactatc

SEQ ID NO: 8 - DNA, 24 bp, synthetic construct
  source 1..24: mol_type = other DNA
aatccctggaccggctggaatttc

SEQ ID NO: 9 - DNA, 24 bp, synthetic construct
  source 1..24: mol_type = other DNA
ttgatgcctggctgtcctggaacc

SEQ ID NO: 10 - DNA, 20 bp, synthetic construct
  source 1..20: mol_type = other DNA
cgtggtgacaagggtgagac

SEQ ID NO: 11 - DNA, 20 bp, synthetic construct
  source 1..20: mol_type = other DNA
taggtgatgttctgggaggc

SEQ ID NO: 12 - protein, 114 aa, synthetic construct
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLKEVIQKILDSGNKKN

SEQ ID NO: 13 - protein, 114 aa, synthetic construct
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLKKVIQEILDSGNKKN

SEQ ID NO: 14 - protein, 114 aa, synthetic construct
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLKEVIQEILDSGNKKN

SEQ ID NO: 15 - protein, 114 aa, synthetic construct
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLEEVIQEILDSGNKKN

SEQ ID NO: 16 - protein, 114 aa, synthetic construct
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLEEVIQKILDSGNKKN

SEQ ID NO: 17 - protein, 114 aa, synthetic construct
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLEKVIQEILDSGNKKN

SEQ ID NO: 18 - protein, 114 aa, synthetic construct
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLEKVIQKILDSGNKKN

(19) United States
(12) Patent Application Publication
(10) Pub. No.: US 2026/0092091 A1
(43) Pub. Date: Apr. 2, 2026

(54) CXC RECEPTOR LIGANDS

(71) Applicants: QUEEN MARY UNIVERSITY OF LONDON, London (GB); THE UNIVERSITY OF MANCHESTER, Manchester (GB)

(72) Inventors: Nikolaos KOUVATSOS, Manchester (GB); Anthony DAY, Manchester (GB); Francesco DELL'ACCIO, London (GB); Sara CAXARIA, London (GB)

(21) Appl. No.: 19/120,219

(22) PCT Filed: Oct. 11, 2023
(86) PCT No.: PCT/GB2023/052634

§ 371 Date: Apr. 10, 2025

(30) Foreign Application Priority Data
Oct. 11, 2022 (GB) 2214951.2

(51) Int. Cl. C07K 14/52; A61K 38/00; A61P 19/02

(52) U.S. Cl. CPC C07K 14/521; A61P 19/02; A61K 38/00

(57) ABSTRACT

The present invention relates to novel polypeptide ligands of CXCR1 and/or CXCR2 which are derived from GCP2. The invention also concerns polynucleotides encoding the polypeptide ligands, uses of the polypeptide ligands and compositions comprising the polypeptide ligands.

### FIELD OF THE INVENTION

The present invention relates to novel polypeptide ligands of CXCR1 and/or CXCR2 which are derived from GCP2. The invention also concerns polynucleotides encoding the polypeptide ligands, uses of the polypeptide ligands and compositions comprising the polypeptide ligands.

### BACKGROUND OF THE INVENTION

The CXC chemokine receptor family are bound and activated by CXC chemokines. CXC chemokine receptors are a large family of seven-transmembrane G-protein coupled receptors that are often expressed on the surface of inflammatory cells, e.g. leukocytes, and are mediators of inflammation.

The CXC chemokine receptor family members, CXCR1 and CXCR2, are known to be expressed in cartilage. Upon binding and activation of CXCR1 and CXCR2 by their respective ligands (CXC chemokines), the receptors are internalized, consequently initiating a signalling cascade that evokes intracellular calcium mobilization, activation of the AKT pathway, and cytoskeletal reorganization.

CXC chemokine mediated inflammation is in part evoked by the chemokines acting as chemoattractants with respect to inflammatory cells, subsequently enabling their binding and activation of the CXC chemokine receptors on the surface of inflammatory cells. CXC chemokine mediated inflammation is concurrently affected by the ability of CXC chemokines to bind glycosaminoglycans (GAGs) on the internal surface of blood vessels, thus attracting inflammatory cells and facilitating their trans-endothelial migration to sites of inflammation.

### SUMMARY OF THE INVENTION

The polypeptide ligands of the present invention are expected to be particularly effective in binding and activating CXCR1 and/or CXCR2 whilst simultaneously reducing leukocyte attraction. The reduction in leukocyte attraction exhibited by the polypeptide ligands of the invention is the result of the polypeptide ligands' reduced ability to bind glycosaminoglycans (GAGs) compared to wild-type GCP-2 (granulocyte chemotactic protein 2) protein. The polypeptide ligands of the invention comprise at least one substitution of a positively charged amino acid for a negatively charged or neutral amino acid.

An effective therapy for diseases characterised by articular cartilage, bone changes, pain and disability, e.g. osteoarthritis, needs to maintain cartilage homeostasis whilst abrogating any inflammatory immune response in the cartilage. The present inventors have demonstrated that CXCR1 and CXCR2 are key players in promoting cartilage homeostasis by promoting chondrocyte phenotypic stability and articular chondrogenesis, whilst preventing chondrocyte hypertrophy and osteogenesis. Thus, activating these receptors with a ligand such as GCP2 can have beneficial anabolic effects on cartilage. However, the present inventors have also demonstrated that wild-type GCP2 evokes simultaneous chemotaxis whilst activating CXCR1 and CXCR2. This can lead to detrimental inflammation, since it promotes migration and infiltration by cells of the immune system that can have inflammatory effects, which may be referred to collectively herein as “inflammatory cells”. These cells are primarily leukocytes, including particularly neutrophils, but also other leukocyte subsets such as monocytes, mast cells, NK cells and others.

The present inventors have discovered that substituting at least one positively charged amino acid for a neutral or negatively charged amino acid from a ligand of the chemokine receptors CXCR1 and/or CXCR2, or chemically modifying at least one positively charged amino acid of the ligand, can maintain the ligand's ability to bind and activate CXCR1 and/or CXCR2, thus maintaining cartilage homeostasis, whilst simultaneously reducing chemotaxis by reducing the ligand's ability to bind GAGs as compared to wild-type GCP2. The polypeptide ligands of the invention therefore represent novel therapies that are suitable for a wide variety of treatment regimes, particularly the treatment of osteoarthritis.

The invention provides a polypeptide ligand of chemokine receptor CXCR1 and/or CXCR2, which:
a. has an ability to activate CXCR1 and/or CXCR2; and
b. has a reduced ability to bind to glycosaminoglycans (GAGs) as compared to GCP2 of SEQ ID NO: 1;
   wherein said polypeptide comprises or consists of the sequence of SEQ ID NO: 1 in which at least one positively charged amino acid has been substituted for a negatively charged or neutral amino acid, or in which at least one positively charged amino acid has been chemically modified to neutralise the positive charge.

The invention also provides a polynucleotide which encodes a polypeptide ligand of the invention.

The invention also provides a composition comprising a polypeptide ligand of the invention and/or a polynucleotide of the invention, which comprises at least one pharmaceutically acceptable diluent, carrier, preservative or excipient.

The invention also provides a method of treating or preventing a disease or condition in a subject, the method comprising administering to the subject a polypeptide ligand of the invention, a polynucleotide of the invention, and/or a composition of the invention.

### BRIEF DESCRIPTION OF THE FIGURES

FIG. 1 shows GCP-2 is expressed in the prospective permanent articular cartilage in embryonic development and in adult cartilage. A Immunostaining of a murine knee joint at 18.5 dpc with an anti GCP-2 or IgG control antibody. B Immunohistochemistry for GCP-2 (red) in the knee of a human 5-month old fetus. C, D Immunostaining for GCP-2 in an adult (12 week old) mouse knee (C) or in adult human articular cartilage (D). E=epiphyseal cartilage; A=articular cartilage; M=meniscus; T=tibia; F=femur; T-AC=tibial articular cartilage; F-AC=femur articular cartilage; in C, dotted lines indicate the osteochondral boundary and the cartilage surface and the arrow the bone marrow spaces.

FIG. 2 shows GCP-2 supports the stable chondrocyte phenotype and chondrogenic differentiation in vitro and in vivo. A-C GAG content as assessed by Alcian blue staining with densitometry (A) or spectrophotometric (B-C) quantitation. A HAC micromasses treated with a GCP-2 neutralizing antibody (n=4) or IgG control (n=4); p values were determined by the Mann-Whitney U test. B C28/I2 micromasses treated with GCP-2 siRNA (n=12) or scrambled control siRNA (Scr; n=14); p values were determined by unpaired two-tailed Student's t test. C C3H10T½ micromasses treated for 3 days with recombinant GCP-2 100 ng/ml (n=4) or vehicle (n=4) or BMP-2 (n=4); p values were determined by ANOVA followed by Tukey HSD post hoc test. D qPCR for ACAN (Aggrecan) expression in C3H10T½ micromasses treated for 3 days with recombinant GCP-2 (100 ng/ml) (n=3) or vehicle (n=4); ACAN—Aggrecan; p values were determined by unpaired two-tailed Student's t test. E spectrophotometric quantification of GAG release (dimethylmethylene blue assay) in supernatant of human cartilage explants treated with recombinant GCP-2 (n=4) or vehicle (n=4) and incubated for 24h, p values were determined by Mann-Whitney U test. F Toluidine blue staining of ectopic cartilage explants collected 2 weeks after subcutaneous co-implantation of HACs in a 10:1 ratio with growth-arrested COS7 expressing either GFP (n=3) or GCP-2 (n=3) and quantification—on the right hand side—of the metachromatically stained area as % of total area; scale bar=200 μm; p values were determined by unpaired two-tailed Student's t test. G Collagen 2 (Col2) immunostaining (left) and quantification (right) as % of total area; scale bar=100 μm; p values were determined by unpaired two-tailed Student's t test.

FIG. 3 shows GCP-2 prevents hypertrophic differentiation and calcification of chondrocytes A, B qPCR for (A) Runx2 and (B) Col1A1 expression in HAC micromasses treated for 24 hours with recombinant GCP-2 (100 ng/ml) or vehicle (n=4); Runx2=Runt-related transcription factor 2, Col1A1 collagen type 1 alpha 1; p values were determined by unpaired two-tailed Student's t test. C Alkaline phosphatase staining (red) in HAC monolayer after 3 week treatment with T3 hormone (100 ng/ml), T3 hormone+GCP-2 (100 ng/ml), or control medium; scale bar 100 μm. D quantification of alkaline phosphatase stained area (n=3); p values were determined by fitting a generalised linear model followed by pairwise comparison of the estimated marginal means. E qPCR for Col10A1 (n=3) of HAC 3 weeks after treatment with vehicle, T3 (100 ng/ml) or T3+GCP-2 (100 ng/ml); n=3; p values were determined by ANOVA with Tukey's HSD post-hoc test. (F-H) C3H10T½ monolayers were treated with osteogenic medium (OM) or OM+GCP-2 (100 ng/ml): (F) number of cells per well positive for alkaline phosphatase staining (n=3 or n=4), (G) number of alkaline phosphatase positive nodules per well (n=7), and (H) spectrophotometric quantification of alizarin red staining at 570 nm (n=6); p values were determined by unpaired, two-tailed Student's t test.

FIG. 4 shows Disrupting GCP-2 GAG binding dissociates the chemotactic from the chondrogenic effects of GCP-2 A Human GCP-2 sequence aligned with closely related human CXCL proteins and the mouse CXCL5 (mCXCL5). The secondary structure is predicted based on the NMR structure of human CXCL5 (PDB: 2MGS). Conserved residues are highlighted in red boxes (white font) and partially conserved residues with similar chemical properties are shown in red font. B, C GAG binding capacity of wildtype GCP-2, and mutants (K101E single mutant, K105E single mutant, GCP-2-D=K101E_K105E double mutant and GCP-2-T=triple mutant K100E_K101E_K105E) was assessed by (B) a heparin microtiter plate binding assay measuring the interaction of biotinylated-heparin (b-heparin) with immobilized proteins and (C) heparin affinity chromatography equilibrated and run in PBS, pH 7.4. In (C) the proteins are eluted with a salt gradient from 0-2 M NaCl monitored by the conductivity (Cond), where 20 and 90 mS/cm correspond to 193 and 1360 mM NaCl, respectively; GCP-2-T, GCP-2-D, K101E, K105E and WT proteins elute at 530 mM, 617 mM, 815 mM, 850 mM and 1024 mM NaCl, respectively. D,E Maximum migratory activity of WT GCP-2 (50 nM) was compared to GCP-2 mutants (50 nM) in (D) chemotaxis and (E) trans-endothelial migration assays in CXCR2-expressing 300-19 pre B cells. WT=wildtype GCP-2; K101E=GCP-2_K101E single mutant; K105E-GCP-2_K105E single mutant; D-GCP-2_K101E_K105E double mutant; T-GCP-2_K100E_K101E_K105E triple mutant; p values were determined by fitting a generalised linear model followed by pairwise comparison of the estimated marginal means. F-H C28/I2 micromasses stimulated with WT GCP-2, GCP-2 triple mutant (GCP-2-T) or untreated and assessed for: (F) AKT phosphorylation levels by western blotting: V=vehicle treated samples, GCP-2=WT GCP-2 treated samples, GCP-2-T=triple mutant GCP-2 treated samples, pAKT=phosphorylated AKT, tAKT=total AKT. On the right, quantification of the density of the bands using ImageJ. Five independent experiments were analyzed. The data were scaled and p values were determined by fitting a generalised linear model followed by pairwise comparison of the estimated marginal means. G spectrophotometric quantification of proteoglycan content by Alcian blue staining. p values were determined by Kruskal-Wallis test with Dunn's post-hoc test; (n=8) H ACAN expression levels by qPCR; ACAN=Aggrecan; p values were determined by ANOVA with Tukey's HSD post-hoc test; n=4. I-J Real time quantitative PCR of COL2A1 (I) and COL10A1 (J) mRNA normalized to β actin. p values were determined by fitting a generalised linear model followed by comparison of the estimated marginal means; n=5 for 5 COL2A1 and 6 for COL10A1.K Number of neutrophils counted within the intercondylar space on sections from mice killed 4 days after an intra-articular injection of adenovirus encoding GFP (n=3), GCP-2 (n=3) and GCP-2-T (n=3); p values were determined by fitting a generalised linear model (family=poisson) followed by pairwise comparison of the estimated marginal means.

FIG. 5 shows Exogenous GCP-2-T improves structural outcomes and pain in osteoarthritis. A Schematic of in vivo experimental osteoarthritis. B Weekly pain measurement by incapacitance, shown as the percentage of bodyweight loaded on the operated leg in GFP (n=15) vs GCP-2 (n=14) and GFP vs GCP-2-T (n=15) treated mice. Circles show the mean, and error bars show 95% confidence intervals. P values were determined by building a mixed-effect linear model. C Incapacitance at week 10 (final time-point); red line indicates 50% loading (no pain); p values were determined by unpaired, two-tailed one sample Student's t test testing the hypothesis that mice loaded 50% body weight on the operated limb. D The area under the curve (AUC) of incapacitance calculated starting from week 6; p values were determined by ANOVA with Tukey's HSD post-hoc test. E, F Osteoarthritis severity assessed using (E) OARSI scoring system 10 weeks after MLI; GFP (n=7), GCP-2 (n=7) and GCP-2-T (n=7); p values were determined by fitting a generalized linear model followed by comparison of the estimated marginal means. F representative image (Safranin O staining) for each treatment; arrows indicate the tide-mark; scale bar 100 μm. G Quantification of osteophytes area; GFP (n=7), GCP-2 (n=7) and GCP-2-T (n=8); p values were determined by ANOVA with Tukey's HSD post-hoc test. H representative images with osteophyte highlighted; scale bar 300 μm. I osteophyte maturity score (n=7); p values were determined by fitting a generalized linear model followed by pairwise comparison of the estimated marginal means.

FIG. 6 shows siRNA validation in vitro. Effect of GCP-2 on AKT phosphorylation in chondrocytes and on GAG production in HACs. A-B C28/I2 (A) or C3H10T½ (B) monolayers transfected with 25 nM of either scrambled or GCP-2 siRNA for 72h and analysed for GCP-2 levels by western blotting. C C28/I2 micromasses stimulated for 3 days with recombinant GCP-2, IGF-1 (positive control) or untreated (vehicle) and assessed for AKT phosphorylation levels by western blotting; β-actin as loading control; GCP-2=WT, GCP-2 treated samples, IGF-1=IGF-1 treated samples, V=vehicle treated samples, pAKT=phosphorylated AKT. D Alcian blue staining and spectrophotometric quantitation of GAGs in micromasses of HACs treated with recombinant GCP-2 or vehicle (n=3); p values were determined by unpaired, two-tailed Student's t test. OD=optical density.

FIG. 7 shows GCP-2 model and characterization of WT and mutants. A 3D homology model of human GCP-2 based on the NMR structure of human CXCL5 dimer (PDB: 2MGS) generated using SWISS-MODEL. The GCP-2 monomers are coloured blue and green with Lys100, Lys101 and Lys105 shown in space filling for the latter. B Comparison of 1D NMR spectra of WT and mutant GCP-2 proteins (all human) made in this study. C SDS-PAGE analysis of GCP-2 WT and mutants under reducing (R) and non-reducing (NR) conditions. Protein samples (1 μg) were either reduced and alkylated (R) or alkylated (NR). MW size from protein markers is displayed on the left. WT=wildtype GCP-2; K101E=K101E GCP-2_single mutant; K105E=K105E GCP-2_single mutant; D=K101E_K105E GCP-2_double mutant; T=K100E_K101E_K105E GCP-2_triple mutant. D, E Dose-dependency of WT GCP-2-induced (D) chemotaxis and (E) and trans-endothelial migration (TME) of CXCR2-expressing 300-19 pre B cell line (mean values±SEM). F Representative images of immunostaining for the Ly6G neutrophil marker in the intercondylar notch of mice injected intraarticularly with GFP, GCP-2, or GCP-2-T adenovirus. Time point: 2 days. White arrowheads indicate neutrophils. G average thickness of the synovial membrane 4 days after the intra-articular injection of adenovirus encoding GFP. GCP-2 or GCP-2-T. The thickness of the synovial membrane was assessed by histomorphometry using ImageJ. P values were determined with ANOVA n=4 per group. H caliper measurement of knee size four days after the injection of GFP, GCP-2 and GCP-2-T adenovirus; p values were determined by one way ANOVA (n=6).

FIG. 8 shows Exogenous GCP-2-T does not affect subchondral bone density and synovium thickness in osteoarthritis. A Bone density of subchondral bone in operated and sham operated knees as accessed by microCT as BV/TV. Mixed effect model followed by pairwise comparison of the estimated marginal means (n=42). B Density of subchondral bone in different treatments as accessed by microCT as BV/TV; GFP (n=7), GCP-2 (n=7) and GCP-2-T (n=6); p values were determined by ANOVA with Tukey's HSD post-hoc test. C Synovium thickness in μm assessed as average thickness of synovium in 6 areas of the joint; GFP (n=7), GCP-2 (n=7) and GCP-2-T (n=8); p values were determined by ANOVA with Tukey's HSD post-hoc test.

FIG. 9 shows GCP-2-T activates AKT phosphorylation in vivo—molecular characterization of osteoarthritis in mice. A Ten-week-old female mice were injected intra-articularly with 6 μl of GFP (n=4), GCP-2 (n=3) or GCP-2-T (n=3) adenovirus and killed 4 days later for immunofluorescence analysis of phospho-AKT (pAKT). After thresholding, pAKT⁺ cells were counted using ImageJ. p values were obtained by fitting a generalized linear model (family=Poisson) followed by pairwise comparison of the estimated marginal means. B-D sections from the MLI experiment in FIG. 5 were used to assess the expression of (B) collagen type II (Col2), (C) collagen type X (Col10), (D) NITEGE neo-epitope using immunofluorescence, and (E) apoptosis using the TUNEL assay; n=4 in the GCP-2 and GCP-2-T groups and 3 in the GFP group; p values were obtained by fitting a generalized linear model. Whenever multiple technical replicates from the same knee were available, individual values were averaged. Scale bar=50 μm.

FIG. 10 shows schematic of GCP-2-T functioning in the cartilage and vascular compartments.

### BRIEF DESCRIPTION OF THE SEQUENCES

SEQ ID NO: 1 is the polypeptide sequence of the human GCP2 (CXCL6) protein (UniProt P80162) that includes the signal peptide and the first amino acid is a methionine.
SEQ ID NO: 2 is Human and Mouse B Actin Forward primer
SEQ ID NO: 3 is Human and Mouse B Actin Reverse primer
SEQ ID NO: 4 is Mouse Aggrecan Forward primer
SEQ ID NO: 5 is Mouse Aggrecan Reverse primer
SEQ ID NO: 6 is Human RUNX2 Forward primer
SEQ ID NO: 7 is Human RUNX2 Reverse primer
SEQ ID NO: 8 is Human Col10A1 Forward primer
SEQ ID NO: 9 is Human Col10A1 Reverse primer
SEQ ID NO: 10 is Human Col1A1 Forward primer
SEQ ID NO: 11 is Human Col1A1 Reverse primer
SEQ ID NO: 12 to 18 are sequences of exemplary molecules of the invention

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polypeptide” includes “polypeptides”, and the like.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogs and peptidomimetics.

The terms “patient” and “subject” are used interchangeably and typically refer to a human.

All publications, patents and patent applications cited herein, whether supra or infra, are hereby incorporated by reference in their entirety.

### Polypeptide

The present inventors have discovered that substituting at least one positively charged amino acid for a neutral or negatively charged amino acid from a ligand of the chemokine receptors CXCR1 and/or CXCR2, or chemically modifying at least one positively charged amino acid of the ligand, can maintain the ligand's ability to bind and activate CXCR1 and/or CXCR2, thus maintaining cartilage homeostasis, whilst simultaneously reducing chemotaxis by reducing the ligand's ability to bind GAGs (glycosaminoglycans) as compared to wild-type GCP2 (granulocyte chemotactic protein 2).

The terms “protein”, “polypeptide”, and “peptide” are interchangeably used further herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like.

The polypeptide of the invention is a chemokine receptor ligand. Chemokine receptors are known in the art and include CXCR1, CXCR2, CXCR3, CXCR4, CXCR5, CXCR6, CXCR7, CCR8, CCR2, CCR1, CCR1, CCR3, CCR5, CCR4, CCR6, CCR7, CCR9, CCR10, XCR1 and CX3CR1. The CXC chemokine receptor family is characterised by their binding of chemokines that have a Cys-X-Cys (CXC) sequence, wherein ‘X’ may be any amino acid, at their N-terminal.

CXC chemokines that bind and activate one or more CXC chemokine receptors are known in the art. The CXC chemokines may be further characterised by having or lacking an ELR motif. An ELR motif is a Glu-Leu-Arg motif that may be present close to the N-terminus of a CXC chemokine, prior to the first cysteine residue in the polypeptide sequence (reading the sequence from its N to C terminus) of the CXC chemokine. Examples of human CXC chemokines that have an ELR motif (ELR+) may include CXCL1, CXCL2, CXCL3, CXCL5, CXCL6 (GCP-2), CXCL7, CXCL8, CXCL15 and CXCL17. Examples of CXC chemokines that do not have an ELR motif (ELR-) may include CXCL4, CXCL9, CXCL10, CXCL11, CXCL12, CXCL13, CXCL14, CXCL16. CXC chemokines may have the ability to bind more than one CXC chemokine receptor. The polypeptide ligand of the invention is a ligand of the chemokine receptors CXCR1 and/or CXCR2. The polypeptide ligand of the invention may additionally be a ligand of any one or more of the CXC chemokine receptor family including CXCR1, CXCR2, CXCR3, CXCR4, CXCR5, CXCR6.

The term “ligand” is used herein to refer to a polypeptide capable of binding, and thus forming a complex with, another biomolecule. Binding may occur via intermolecular forces, such as ionic bonds, hydrogen bonds and Van der Waals forces.

The invention concerns a polypeptide ligand of chemokine receptors CXCR1 and/or CXCR2 which: (i) has an ability to activate CXCR1 and/or CXCR2; and (ii) has a reduced ability to bind to GAGs as compared to GCP-2 of SEQ ID NO: 1; wherein said polypeptide comprises or consists of the sequence of SEQ ID NO: 1 in which at least one positively charged amino acid has been substituted for a negatively charged amino acid, or in which at least one positively charged amino acid has been chemically modified to neutralise the positive charge.

The polypeptide ligand of the invention has an affinity for CXCR1 and/or CXCR2. Binding “affinity” refers to the strength of the binding interaction between the ligand and binding partner e.g. a biomolecule such as a chemokine receptor. The term “affinity” can be used interchangeably with “binding ability” or “ability to bind”. The binding assay used to quantify the affinity of the polypeptide ligands of the invention, and optionally GCP-2, for CXCR1 and/or CXCR2 may be any suitable protein-protein binding assay known in the art. Suitable assays include, but are not limited to, cell-based binding assays using labeled ligands (e.g. fluorescent, radiolabeled), immunoprecipitation and western blotting, enzyme-linked immunosorbent assay (ELISA), fluorescence resonance energy transfer (FRET) and surface plasmon resonance (SPR).

The polypeptide ligands of the invention are ligands of the chemokine receptors CXCR1 and/or CXCR2 and have a reduced ability to bind GAGs as compared to that of GCP-2 of SEQ ID NO: 1. Preferably, the relative affinities of the polypeptide ligand of the invention and GCP2 of SEQ ID NO: 1 for GAGs are quantified by the same assays. The binding assay used to quantify the affinity of the polypeptide ligands of the invention and GCP-2 may be any suitable protein-GAG binding assay known in the art. Suitable assays include, but are not limited to, microtitre plate-based binding assays, affinity chromatography, SPR, isothermal titration calorimetry, NMR spectroscopy, spectroscopic elipsometry, quartz crystal microbalance with dissipation monitoring, biolayer interferometry, microscale thermophoresis, analytical ultracentrifugation, immunoprecipitation and western blotting, enzyme-linked immunosorbent assay (ELISA), and surface plasmon resonance (SPR).

The polypeptide ligand of the invention is a ligand of the chemokine receptors CXCR1 and/or CXCR2 and preferably has at least a 1%, 2%, 3%, 4%, 5%, 10%, 20%, 30%, 40%, 50%, 60%, 65%, 70%, 75%, 80%, 85%, 90% or 95% reduction in ability to bind GAGs as compared to that of GCP-2 of SEQ ID NO: 1. Even more preferably, the polypeptide ligand of the invention is a ligand of the chemokine receptors CXCR1 and/or CXCR2 and is not able to bind GAGs.

As used herein, the term “GAG” may refer to any form of GAG known in the art. Examples of GAGs include chondroitin sulfate, dermatan sulfate, keratan sulfate, heparosan, heparan sulfate, heparin and hyaluronan. The different forms of GAGs have functions that are known in the art. All GAGs typically have a high negative charge density. GAGs may also be present on the walls of blood vessels at inflammatory sites. ELR-CXC chemokines may bind to GAGs present on the walls of blood vessels, consequently attracting inflammatory cells. GAGs may be present on the internal and/or external walls of blood vessels. ELR+ CXC chemokine-mediated attraction of inflammatory cells is a mediated via the binding of ELR+ CXC chemokines to CXC chemokine receptors on the surface of inflammatory cells. Particularly, the inflammatory cells attracted by ELR+ CXC chemokines are leukocytes. The binding of ELR+ CXC chemokines to the CXC receptors of inflammatory cells, particularly leukocytes, may initiate the process of trans-endothelial migration through which inflammatory cells exit the blood vessel and accumulate at the site of inflammation. CXC chemokine binding to GAGs is therefore essential for transendothelial migration of inflammatory cells.

Any potential chemoattractant (including the ligand of the invention) may be assayed for its ability to attract inflammatory cells by any chemotaxis assay known in the art. The chemotaxis assay may be an in vitro or an in vivo assay. GCP2 is a known chemoattractant. The polypeptide ligand of the invention may a have a reduced ability to attract inflammatory cells as compared to GCP2 of SEQ ID NO: 1. It may thus be described as being a weaker chemoattractant than wildtype GCP2. The polypeptide ligand of the invention preferably has a reduced ability to attract inflammatory cells as compared to GCP2 of SEQ ID NO: 1. The polypeptide ligand of the invention may have at least a 1%, 2%, 3%, 4%, 5%, 10%, 20%, 30%, 40%, 50%, 60%, 65%, 70%, 75%, 80%, 85%, 90% or 95% reduction in ability to attract inflammatory cells as compared to that of GCP-2 of SEQ ID NO: 1. Even more preferably, the polypeptide ligand of the invention is not able to attract inflammatory cells.

CXC chemokines bind to and activate CXC receptors. CXC receptor activation involves receptor internalization, calcium mobilization, and activation of the AKT pathway, which in turn evokes cytoskeletal reorganization. Any molecule (including a ligand of the invention) may be assayed for its ability to activate a CXC receptor by any suitable assay known in the art, such as by the assessment of receptor internalization, calcium mobilization, and activation of the AKT pathway, which in turn evokes cytoskeletal reorganization. GCP2 is an example of a CXC chemokine that can bind and activate a CXC receptor. Particularly, GCP2 may bind and activate CXCR1 and CXCR2. The polypeptide ligand of the invention may be capable of activating CXC receptors. Particularly, the polypeptide ligand of the invention may be capable of activating CXCR1 and/or CXCR2.

The invention concerns a polypeptide ligand of chemokine receptors CXCR1 and/or CXCR2 which: (i) has an ability to activate CXCR1 and/or CXCR2; and (ii) has a reduced ability to bind to GAGs as compared to GCP-2 of SEQ ID NO: 1; wherein said polypeptide comprises or consists of the sequence of SEQ ID NO: 1 in which at least one positively charged amino acid has been substituted for a neutral or negatively charged amino acid, or in which at least one positively charged amino acid has been chemically modified to neutralise the positive charge. The ability of the polypeptide ligand to activate CXCR1 and/or CXCR2 may be reduced as compared to that of GCP-2 of SEQ ID NO: 1. Preferably, the ability of the polypeptide ligand to activate CXCR1 and/or CXCR2 is not significantly reduced as compared to that of GCP-2 of SEQ ID NO: 1. Preferably, the ability of the polypeptide ligand to activate CXCR1 and/or CXC2 is substantially the same, or at least the same, as that of GCP-2 of SEQ ID NO: 1. The substitution or chemical modification of at least one positively charged amino acid may reduce GAG binding as compared to GCP-2 of SEQ ID NO: 1. The substitution or chemical modification of further positively charged amino acids may cumulatively further reduce GAG binding as compared to GCP-2 of SEQ ID NO: 1.

The charge status of an amino acid is typically evaluated at pH 7.0. Within the sequence of SEQ ID NO: 1, all of the positively charged amino acids are either lysine (K) or arginine (R). Thus, at least one lysine or arginine in the sequence of SEQ ID NO: 1 may be substituted for a neutral or negatively charged amino acid, or may be chemically modified.

In the polypeptide ligand of the invention, at least one, at least two, at least three, at least four, at least five, at least six, at least seven, at least eight, at least nine, at least ten, at least eleven, at least twelve, or all thirteen of the positively charged amino acids present in wildtype GCP2 may have been substituted for a negatively charged or neutral amino acid, or may have been chemically modified.

Preferably, said positively charged amino acid that is substituted or modified is at least partially responsible for mediating the binding of the GCP2 of SEQ ID NO: 1 to GAGs. Particularly, the positively charged amino acid is preferably at the binding interface between the polypeptide ligand of the invention and GAGs. Thus, the substitution or modification of at least one positively charged amino acid may confer a reduced ability of the polypeptide ligands of the invention to bind GAGs as compared to GCP2 of SEQ ID NO: 1. Said positively charged amino acid may be a lysine, and is preferably K100, K101 or K105. If at least two positively charged amino acids are substituted or modified, said at least two positively charged amino acids are preferably K100 and K101, K100 and K105, or K101 and K105, and are most preferably K101 and K105. If at least three positively charged amino acids are substituted or modified, said at least three positively charged amino acids are preferably K100 and K101 and K105.

In accordance with the invention, a positively charged amino acid may be substituted for a negatively charged or neutral amino acid. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Twenty main amino acids and their respective chemical properties set out in Table 1 below.

TABLE 1
Chemical properties of amino acids at pH 7.0

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | predominantly neutral at pH 7.0 | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

Non-naturally-occurring amino acids, and methods for introducing or substituting non-naturally-occurring amino acids, are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis.

In the polypeptide ligand of the invention described herein, said positively charged amino acid may be substituted with any natural or non-natural amino acid, wherein the polypeptide ligand of the invention (i) has an ability to activate CXCR1 and/or CXCR2; and (ii) has a reduced ability to bind to GAGs as compared to GCP2 of SEQ ID NO: 1. In the polypeptide ligands of the invention described herein, the at least one positively charged amino acid may be substituted with any negatively charged or neutral amino acid, for example selected from: glycine (G), alanine (A), valine (V), leucine (L), isoleucine (I), histidine (H), methionine (M), phenylalanine (F), tryptophan (W), proline (P), serine(S), threonine (T), cysteine (C), tyrosine (Y), asparagine (N), glutamine (Q), aspartic acid (D), glutamic acid (E), or any combination thereof. Said positively charged amino acid may particularly be substituted with be substituted with at least one negatively charged amino acid. Preferred substitutions include the replacement of lysine (K) or arginine (R) with aspartic acid (D) or glutamic acid (E). Replacement of lysine (K) with glutamic acid (E) is particularly preferred. Particular exemplars of this approach are discussed below and are demonstrated in the Example. See also SEQ ID NOs: 12 to 18.

In the polypeptide ligand of the invention, said positively charged amino acid may be at least at K100, wherein K100 is substituted with any negatively charged or neutral amino acid, though more preferably with D or E. More preferably, said positively charged amino acid may be at least at K100, particularly wherein K100 is substituted with E.

In the polypeptide ligand of the invention described herein, said positively charged amino acid may be at least at K101, wherein K101 is substituted with any negatively charged or neutral amino acid, though more preferably with D or E. More preferably, said positively charged amino acid may be at least at K101, particularly wherein K101 is substituted with E.

In the polypeptide ligand of the invention described herein, said positively charged amino acid may be at least at K105, wherein K105 is substituted with any negatively charged or neutral amino acid, though more preferably with D or E. More preferably, said positively charged amino acid may be at least at K105, particularly wherein K105 is substituted with E.

In the polypeptide ligand of the invention described herein, said positively charged amino acid may be at least at K100 and K101, wherein K100 is substituted with any negatively charged or neutral amino acid, though more preferably D or E, and particularly wherein K101 is substituted with any negatively charged or neutral amino acid, though more preferably D or E. Preferably, at least one of K100 and K101 is substituted with E. More preferably, K100 is substituted with E and K101 is substituted with E.

In the polypeptide ligand of the invention described herein, said positively charged amino acid may be at least at K100 and K105, wherein K100 is substituted with any negatively charged or neutral amino acid, though more preferably D or E, and particularly wherein K105 is substituted with any negatively charged or neutral amino acid, though more preferably D or E. Preferably, at least one of K100 and K105 is substituted with E. More preferably, K100 is substituted with E and K105 is substituted with E.

In the polypeptide ligand of the invention described herein, said positively charged amino acid may be at least at K101 and K105, wherein K101 is substituted with any negatively charged or neutral amino acid, though more preferably D or E, and particularly wherein K105 is substituted with any negatively charged or neutral amino acid, though more preferably D or E. Preferably, at least one of K101 and K105 is substituted with E. More preferably, K101 is substituted with E and K105 is substituted with E.

In the polypeptide ligand of the invention described herein, said positively charged amino acid may be at least at K100, K101 and K105, wherein K100 is substituted with any negatively charged or neutral amino acid, though more preferably D or E, wherein K101 is substituted with any negatively charged or neutral amino acid, though more preferably D or E, and wherein K105 is substituted with any negatively charged or neutral amino acid, though more preferably D or E. Preferably, at least one of K100, K101 and K105 is substituted with E. More preferably, at least two of K100, K101 and K105 is substituted with E. Even more preferably, each of K100, K101 and K105 are substituted with an E.

The polypeptide ligand of the invention may be produced by any suitable means. For example, the polypeptide may be synthesised directly using standard techniques known in the art, such as Fmoc solid phase chemistry, Boc solid phase chemistry or by solution phase peptide synthesis. The polypeptide ligand of the invention may be produced using D-amino acids, L-amino acids, or a combination thereof. In the polypeptide ligands of the invention described herein, at least one positively charged amino acid has been substituted for a negatively charged or neutral amino acid, wherein the negatively charged or neutral amino acid is a D-amino acid, an L-amino acid, or a combination thereof.

The polypeptide ligand of the invention may be produced by transforming a cell, typically a bacterial cell, with a nucleic acid molecule or vector which encodes said polypeptide ligand. The invention provides nucleic acid molecules and vectors which encode a polypeptide ligand of the invention. The invention also provides a host cell comprising such a nucleic acid or vector.

Also in accordance with the invention, a positively charged amino acid may be chemically modified. The chemical modification may preferably neutralise the positive charge of the modified amino acid. The chemical modification may confer a reduced ability of the polypeptide ligands of the invention to bind GAGs as compared to GCP2 of SEQ ID NO: 1. Methods for the chemical modification of amino acids are well known in the art. For example, lysine or arginine may be modified by succinylation, or by the application of DEPC, or by PEGylation. Arginine may be modified by application of p-Hydroxyphenylglyoxal. Preferably at least one of K100, K101 and K105 is chemically modified. More preferably at least two of K100, K101 and K105 are chemically modified. Most preferably all three of K100, K101 and K105 are chemically modified.

### Polynucleotides

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment. messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences, for example in an expression vector. The boundaries of the coding sequence are determined by a start codon at 5′ (amino) terminus and a translation stop codon at 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides can be synthesised according to methods well known in the art, as described by way of example in Sambrook et al (1989, Molecular Cloning—a laboratory manual; Cold Spring Harbor Press). The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al.

The invention also includes cells that have been modified to express a polypeptide ligand of the invention. Such cells typically include prokaryotic cells such as bacterial cells, for example E. coli. Such cells may also include eukaryotic cells. Such prokaryotic or eukaryotic cells may be cultured using routine methods to produce a polypeptide ligand of the invention.

The polypeptide ligand of the invention may be in a substantially isolated form. It may be mixed with carriers, preservatives, or diluents (discussed below) which will not interfere with the intended use, and/or with an adjuvant (also discussed below) and still be regarded as substantially isolated. It may also be in a substantially purified form, in which case it will generally comprise at least 90%, e.g. at least 95%, 98% or 99%, of the polypeptide ligand in the preparation.

Particularly, provided by the invention described herein are polynucleotide sequences which encode a polypeptide ligand of the invention. The polynucleotide sequence is preferably any polynucleotide sequence that encodes a polypeptide sequence that comprises or consists of SEQ ID NO: 1 except in that at least one positively charged amino acid of SEQ ID NO: 1 has been substituted for a negatively charged or neutral amino acid. The polynucleotide sequence may comprise any degenerate sequences that encode a polypeptide sequence that comprises or consists of SEQ ID NO: 1 except in that at least one positively charged amino acid of SEQ ID NO: 1 has been substituted for a negatively charged or neutral amino acid.

### Compositions

In another aspect, the present invention provides a composition comprising a polypeptide ligand and/or polynucleotide of the invention. The composition may additionally include at least one diluent, carrier, preservative or excipient. The diluent, carrier, preservative or excipient are each preferably pharmaceutically acceptable. The carrier, preservative and excipient must be ‘acceptable’ in the sense of being compatible with the other ingredients of the composition and not deleterious to a subject to which the composition is administered. Typically, all components and the final composition are sterile and pyrogen free. The composition may be a pharmaceutical composition.

The carrier may be any suitable carrier known to a person skilled in the art. The carrier may be a protein or other suitable substance. The carrier may be linked to the polypeptide of the invention via any suitable method. Carrier proteins include keyhole limpet hemocyanin, serum proteins such as transferrin, bovine serum albumin, human serum albumin, thyroglobulin or ovalbumin, immunoglobulins, or hormones, such as insulin or palmitic acid. Alternatively, the carrier protein may be tetanus toxoid or diphtheria toxoid. Alternatively, the carrier may be a dextran such as sepharose. The carrier must be physiologically acceptable to humans and safe. Preferred carriers include those which improve serum half-life or stability of the polypeptide of the invention. Particularly preferred examples include albumin, polyethylene glycol (PEG) or other polymers, and the Fc region of human immunoglobulin G. Said Fc region may be modified relative to wildtype to further improve serum half-life, for example by incorporation of the YTE and LS mutations.

If the composition comprises an excipient, it must be ‘pharmaceutically acceptable’ in the sense of being compatible with the other ingredients of the composition and not deleterious to the recipient thereof. Auxiliary substances, such as wetting or emulsifying agents, pH buffering substances and the like, may be present in the excipient. These excipients and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Formulation of a suitable composition can be carried out using standard pharmaceutical formulation chemistries and methodologies all of which are readily available to the reasonably skilled artisan. Such compositions may be prepared, packaged, or sold in a form suitable for bolus administration or for continuous administration. Injectable compositions may be prepared, packaged, or sold in unit dosage form, such as in ampoules or in multi-dose containers optionally containing a preservative. Compositions include, but are not limited to, suspensions, solutions, emulsions in oily or aqueous vehicles, pastes, and implantable sustained-release or biodegradable formulations. In one embodiment of a composition, the active ingredient is provided in dry (for e.g., a powder or granules) form for reconstitution with a suitable vehicle (e. g., sterile pyrogen-free water) prior to administration of the reconstituted composition. The composition may be prepared, packaged, or sold in the form of a sterile injectable aqueous or oily suspension or solution. This suspension or solution may be formulated according to the known art, and may comprise, in addition to the active ingredient, additional ingredients such as the adjuvants, excipients and auxiliary substances described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. Alternatively, the active ingredients of the composition may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

### Methods of Use

The polypeptide ligand, polynucleotide or composition of the invention may be used in a method of treating or preventing a disease or condition in a subject. The polypeptide ligand, polynucleotide or composition of the invention may be used in the manufacture of a medicament for use in a method of treating or preventing a disease or condition in a subject. The method comprises administering to the said subject the polypeptide ligand, polynucleotide or composition of the invention. Administration may be of a therapeutically or prophylactically effective quantity of the said polypeptide ligand or the said composition, to a subject in need thereof.

The skilled person would understand that by binding and activating CXCR1 and/or CXCR2, whilst having reduced binding to GAGs, the polypeptide ligand of the invention may promote cartilage homeostasis by promoting chondrocyte phenotypic stability and articular chondrogenesis. Simultaneously, by exhibiting a reduced ability to bind GAGs as compared to wild-type GCP2, the polypeptide ligand of the invention reduces chemotaxis of inflammatory cells and subsequent inflammation in joints. Diseases and conditions whereby the effects of the polypeptide of the invention would be advantageous are well known in known in the art.

The disease or condition may be characterized by pain or stiffness, especially in the hip, knee, and thumb joints. The disease or condition may alternatively, or in addition, be characterised by the degeneration of cartilage, damage to cartilage, or a need for cartilage regeneration, especially in these joints. The disease may be further characterized by hypertrophic differentiation, chondrocyte hypertrophy, calcification and/or bone formation. These characteristics may be directly observed in the patient prior to treatment.

The disease is preferably osteoarthritis. Osteoarthritis is essentially a degenerative disease, where cartilage is destroyed largely due to pathological biomechanics. The ability of the polypeptide ligand of the invention to induce generation of articular cartilage would therefore be directly beneficial in the treatment of osteoarthritis. Additional benefit results because the polypeptide of the invention has reduced binding to GAGs, and hence has a reduced chemotactic attraction for cells of the immune system such as neutrophils, whilst competing with pro-inflammatory chemokines that would otherwise actively promote migration and infiltration of such cells to the joints. This may be particularly beneficial in osteoarthritis because, although it is not directly a disease of inflammation, localised inflammation of the joints may arise in osteoarthritis patients. This inflammation contributes to associated symptoms such as pain, and may also accelerate disease progression because chondrocytes expressing CXCR2 (which might otherwise help to induce articular cartilage regeneration) are susceptible to apoptosis when induced by inflammatory cytokines such as IL-1. Thus, the ability of the polypeptide of the invention to induce regeneration of articular cartilage without promoting migration and infiltration by inflammatory cells (and potentially reducing the pro-infiltration effects of other chemokines) means that it may be particularly advantageous in the treatment of osteoarthritis.

The disease may be late stage inflammatory arthritis such as rheumatoid or psoriatic arthritis; intervertebral disc regeneration, tracheomalacia and also non-unions, where fractures do not heal because a cartilage callus does not form. In each case, there is a need for cartilage regeneration.

When the disease or condition is characterized by pain, the pain may be nociceptive and/or neuropathic pain.

Separately, by occupying the CXCR1 and/or CXCR2 receptors, whilst having reduced binding to GAGs, the polypeptide of the invention may reduce the pro-infiltration/pro-inflammatory effects of chemokines such as CXCL8 and CXCL1 which otherwise bind to CXCR1 and/or CXCR2 receptors. The polypeptide may thus be suitable for treating diseases or conditions in which excessive and/or undesirable inflammation is mediated in whole or in part by these chemokines. Such diseases or conditions are known in the art and may include peritonitis and colitis.

The method may comprise simultaneous or sequential administration with an additional therapy. Suitable additional therapies may depend on the particular disease or condition in the subject. Suitable additional therapies are known in the art. The additional therapy may particularly be an anti-inflammatory agent.

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

### Example

### Introduction

Osteoarthritis is a chronically disabling joint disease characterized by cartilage breakdown, often associated with thickening of the subchondral bone, new bone formation (osteophytes), ligament damage and low-grade inflammation. It causes joint pain, loss of mobility, affects up to a third of the population over the age of 45 and costs around 1.5-2% of GDP. Despite its high prevalence, there are no pharmacological interventions that can arrest or revert progression of cartilage breakdown and avoid the need for joint replacement surgery.

Cartilage abundant extracellular matrix (ECM) is rich in glycosaminoglycans (GAGs) and collagens, which provide load-bearing and tensile strength, respectively.

During development, long bones are initially made of cartilage. Subsequently, chondrocytes in the central part of the skeletal elements (diaphysis) become hypertrophic—expressing collagen type X and other markers—and are eventually replaced by bone. This process, called endochondral bone formation, spares the last few layers of cells proximal to the joint which are resistant to hypertrophic differentiation, vascular invasion, calcification and bone formation and give rise to the permanent articular cartilage. During osteoarthritis, however, ectopic and pathological chondrocyte hypertrophy and calcification take place within the articular cartilage and drive its breakdown.

The inventors showed that the chemokine receptors CXCR1 and CXCR2, and their ligands are expressed in cartilage and contribute to cartilage homeostasis by an autocrine/paracrine mechanism that, through activation of AKT signaling, culminates in upregulation of the cartilage transcription factor SOX9 and of its target genes COL2A1 and ACAN. ELR⁺ CXC chemokines are characterized by a glutamic acid-leucine-arginine (ELR) motif. These chemokines bind and activate the seven-transmembrane G protein-coupled receptors CXCR1 and CXCR2, promoting inflammation through their ability to recruit and activate leukocytes. During inflammation, ELR⁺ CXC chemokines bind to GAG chains present on the blood vessels at inflammatory sites thereby forming a haptotactic gradient on the endothelial surface. The engagement of the chemokines bound to the endothelial surface and the chemokine receptor on the membrane of leukocytes initiates the process of trans-endothelial migration through which leukocytes exit the blood vessel and accumulate in tissues at the site of inflammation. GAG binding, therefore, is essential for trans-endothelial migration of leukocytes.

Here the inventors show that disrupting the capacity of the CXCR2 ligand granulocyte chemotactic protein 2 (GCP-2, also known as-CXCL5 in mice and CXCL6 in humans) to bind GAGs abrogates its pro-inflammatory effects, while preserving its capacity to support cartilage homeostasis; in an experimental osteoarthritis model in mice, GCP-2-T reduced pain and improved cartilage integrity.

### Results

### GCP-2 is Expressed in the Prospective Permanent Articular Cartilage in Embryonic Development and in Adult Cartilage

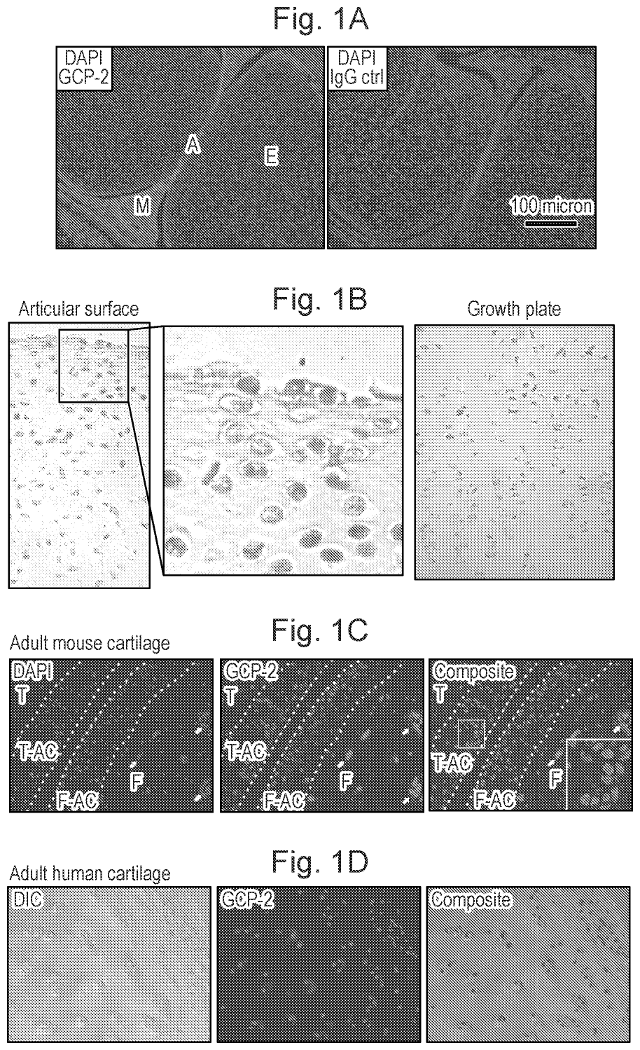

To explore a possible function of GCP-2 in cartilage biology the inventors first investigated its expression pattern during embryonic skeletal development. GCP-2 was detected within the prospective articular cartilage, but not in the epiphyseal cartilage in mice and humans (FIG. 1A-B). GCP-2 staining was retained also in adulthood in both species (FIG. 1C-D).

This expression pattern during development is similar to that of the markers of prospective joint interzones GDF5 and WNT9A—with expression well before the immune system is developed, suggesting that GCP-2 may play a role in cartilage biology independent of its role in inflammation.

### GCP-2 Supports the Articular Chondrocyte Phenotype and Chondrogenic Differentiation

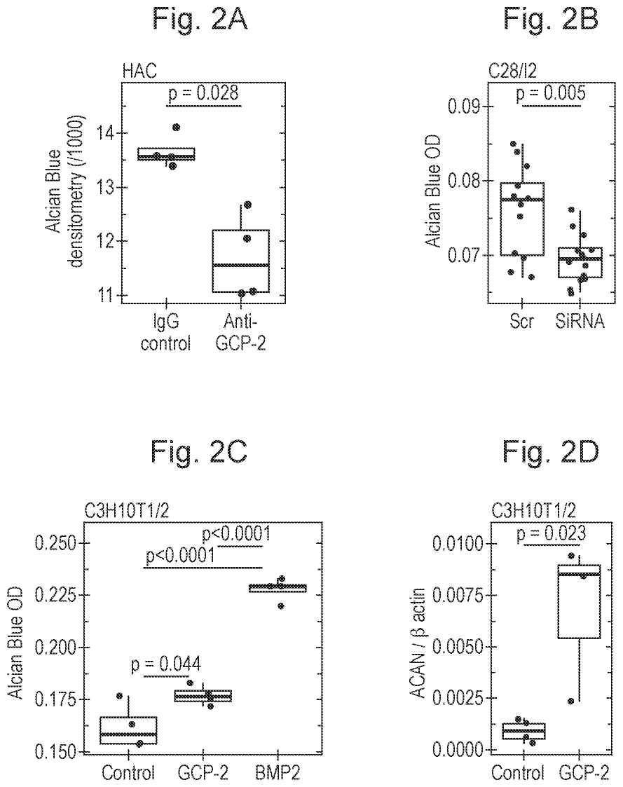

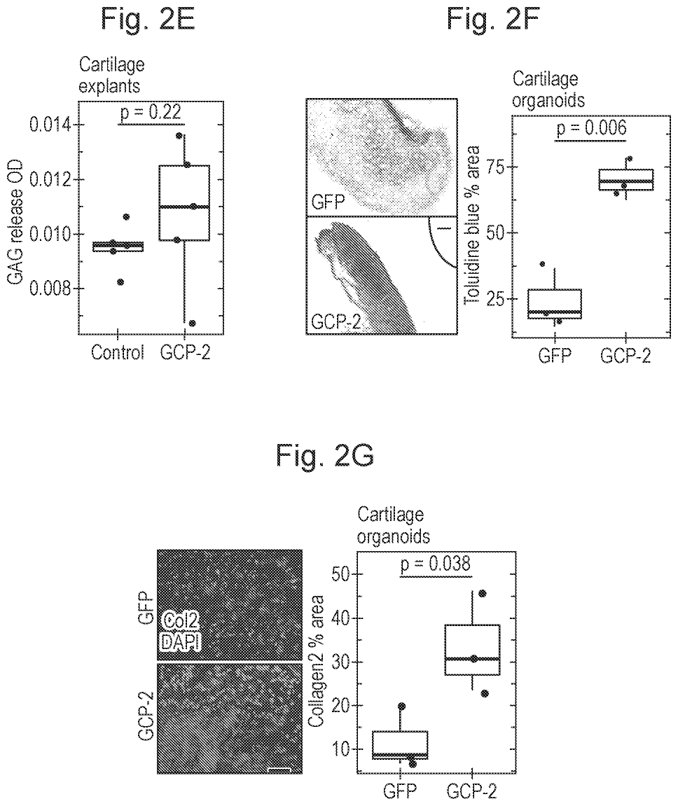

Articular chondrocytes express both GCP-2 and its receptor(s) CXCR1/2 suggesting autocrine/paracrine signaling. To investigate whether endogenous GCP-2 is required to maintain the stable chondrocyte phenotype, we blocked endogenous GCP-2 in primary human articular chondrocytes (HAC) using an anti-GCP-2 neutralizing antibody. After 24 hours, GCP-2 blockade resulted in GAG loss as assessed by Alcian blue staining (FIG. 2A). Silencing GCP-2 with siRNA (˜75% efficiency) led to GAG loss in the human chondrogenic cell line C28/I2 (FIG. 2B).

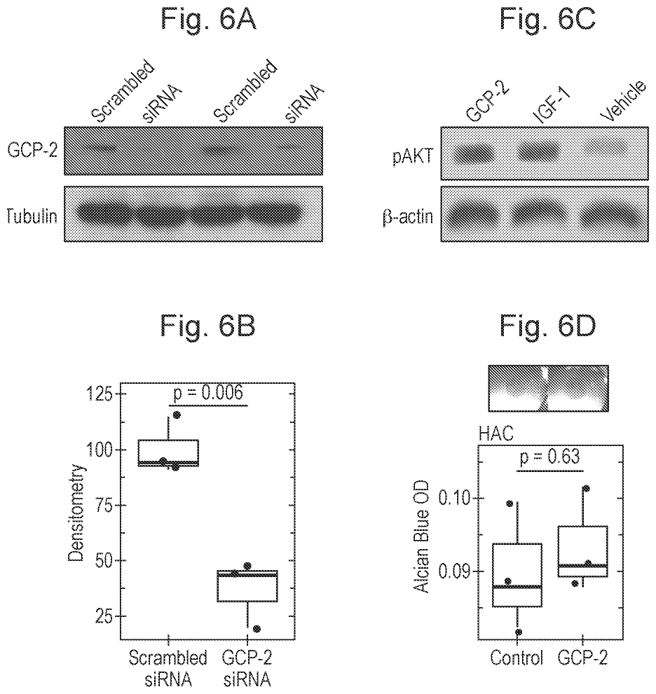

Next, we investigated whether exogenous GCP-2 is sufficient to induce chondrogenic differentiation. Treatment of C3H10T½ micromasses with recombinant GCP-2 (100 ng/ml for 3 days) resulted in accumulation of GAG-rich ECM as measured by Alcian blue staining, albeit to a lesser extent than as induced by BMP-2 (FIG. 2C). It also resulted in an increase of AKT phosphorylation (FIG. 6C)—a known signaling event downstream of CXCR1/2 activation in chondrocytes.

We then investigated whether the effects of GCP-2 treatment on ECM proteoglycan content were due to increased synthesis or reduced catabolism of ECM components. Treatment of C3H10T½ cells with GCP-2 upregulated the expression of aggrecan mRNA (ACAN) (FIG. 2D), a major proteoglycan in cartilage suggesting an anabolic function. Furthermore, treatment of human cartilage explants with GCP-2 did not decrease GAG release in the supernatant as assessed by dimethylmethylene blue assay (FIG. 2E), as would have been expected in the case of reduced catabolism. Taken together these data suggest that the increase in ECM induced by GCP-2 is due to an anabolic effect.

Although GCP-2 blockade resulted in reduced ECM formation in HACs (FIG. 2A), no significant increase in ECM content was detected when fully mature HACs were treated with exogenous GCP-2 (FIG. 6D), possibly owing to their high endogenous GCP-2 expression or ceiling effect.

To test if GCP-2 supports stable cartilage formation in vivo, we used an adoptive model in which HACs implanted ectopically in immunodeficient mice form stable human cartilage organoids resistant to vascular invasion, calcification, and endochondral bone formation.

To deliver exogenous GCP-2 to the implanted HACs for the entire duration of the assay we used a previously validated strategy based on co-implantation of chondrocytes with growth-arrested, GCP-2-overexpressing COS7 cells, or, as control, with GFP-overexpressing COS7 cells. Two weeks after implantation, GCP-2-supplemented HAC implants displayed enhanced ECM formation as determined by increased area of metachromatic Toluidine Blue staining (FIG. 2F), and immunostaining for collagen type II (FIG. 2G).

### GCP-2 Inhibits Hypertrophic Differentiation and Calcification of Articular Chondrocytes

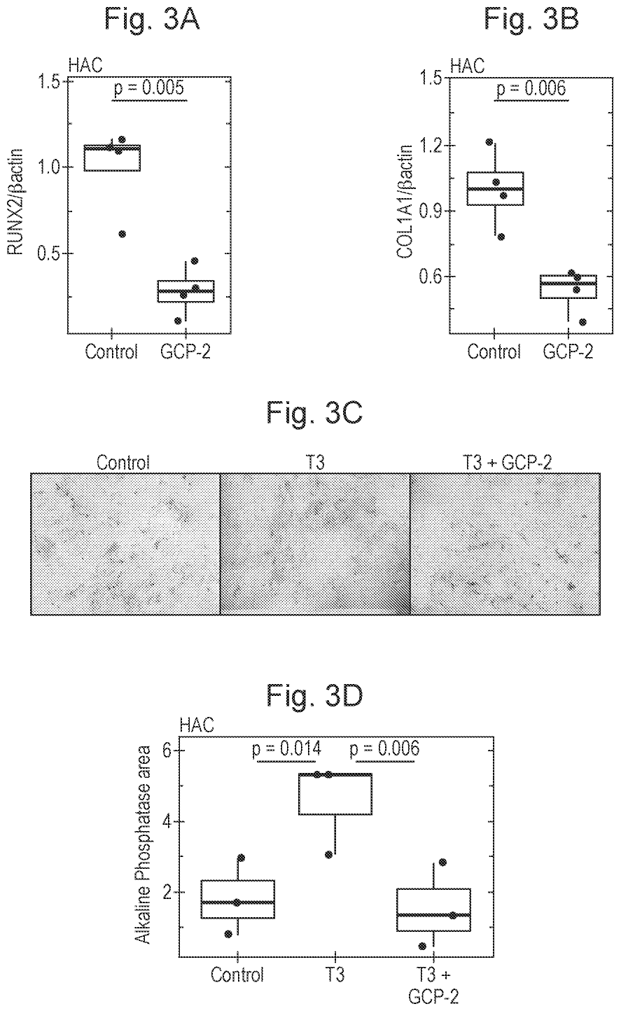

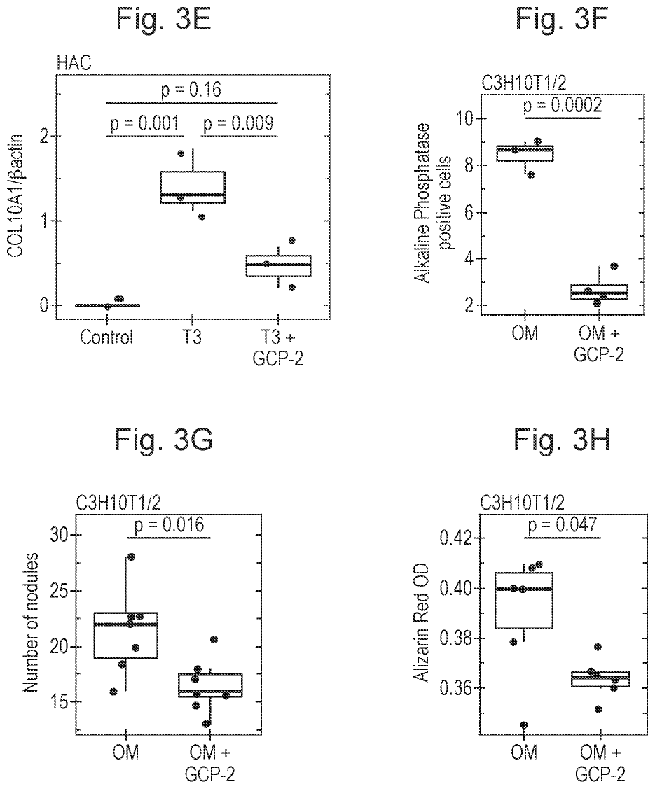

The expression pattern of GCP-2 in development, led us to hypothesize that GCP-2 might prevent hypertrophic differentiation and calcification, a phenomenon driving osteoarthritis progression. Consistent with this hypothesis, exogenous GCP-2 reduced the expression of the hypertrophy marker Runx2 and of the osteogenic marker Col1A1 mRNA in HAC micromasses (FIGS. 3A and 3B).

To further investigate this phenomenon, we tested whether GCP-2 could counteract the capacity of thyroid hormone (T3) to promote hypertrophic chondrocyte differentiation. Consistent with this, we found that, in HAC, GCP-2 blocked the capacity of T3 to induce alkaline phosphatase activity (FIGS. 3C and 3D), and COL10A1 (FIG. 3E), two markers of chondrocyte hypertrophy.

GCP-2 also reduced the capacity of osteogenic medium to induce osteogenesis in C3H10T½ cells as measured by a reduction of the number of alkaline phosphatase positive cells (FIG. 3F) and calcified nodules as measured by Alizarin red staining (FIG. 3G number of nodules and FIG. 3H spectrophotometric quantification).

Disrupting GCP-2 GAG Binding Dissociates the Chemotactic from the Chondrogenic Effects of GCP-2

Although GCP-2 has a beneficial effect on cartilage homeostasis, its pro-inflammatory properties would make it unsuitable as a therapeutic molecule for osteoarthritis. Therefore, we set out to dissociate the pro-inflammatory from chondrogenic effects of GCP-2.

Endothelial cells display GAGs on their surface, to which ELR CXC chemokines bind, creating a haptotactic gradient. Neutrophils, displaying chemokine receptors, interact with and are activated by the chemokines immobilized on the endothelial surface and this interaction initiates transendothelial migration.

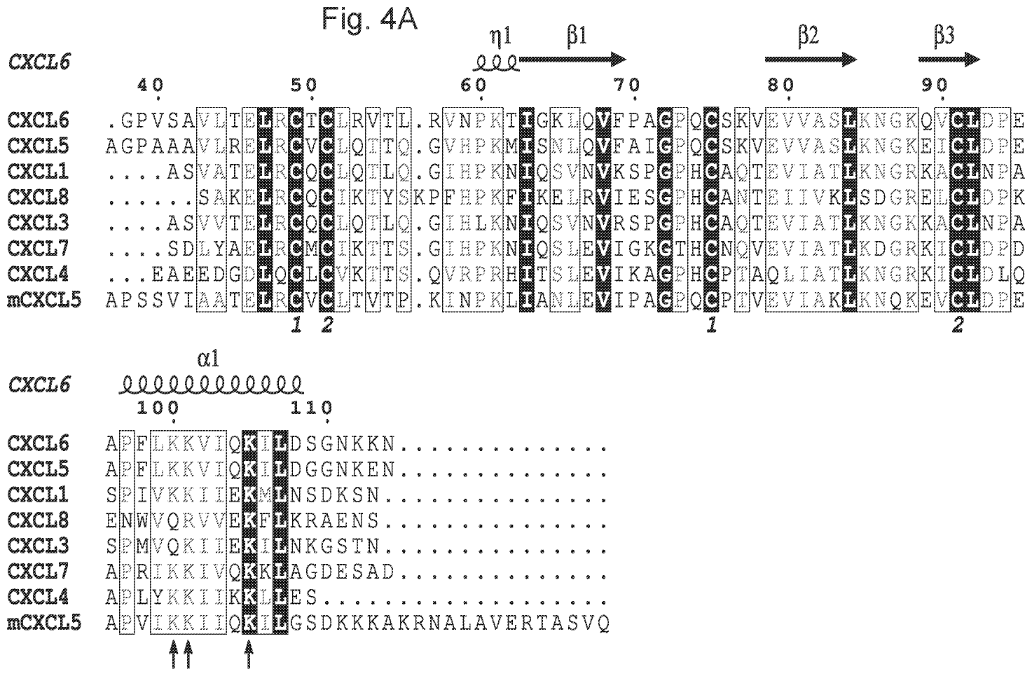

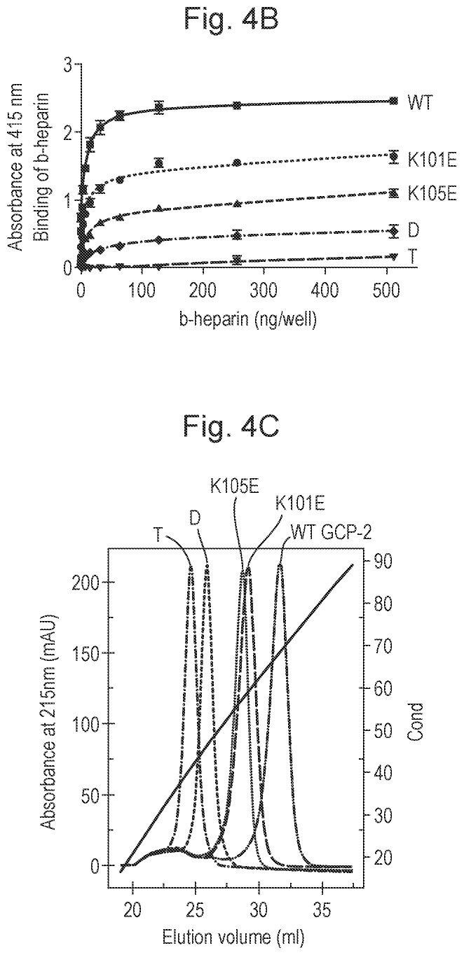

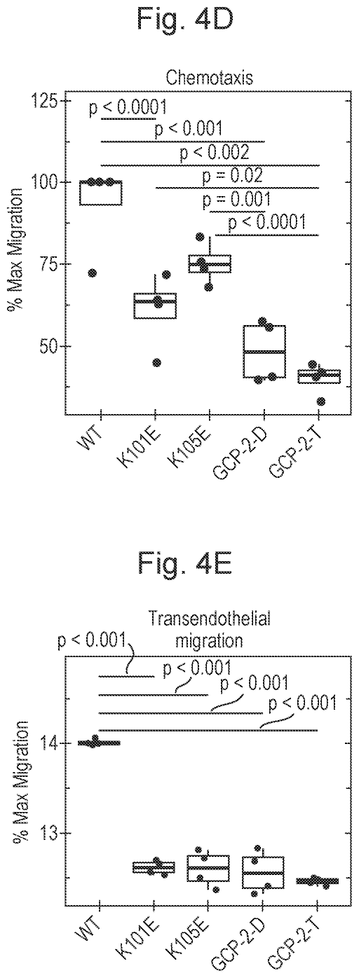

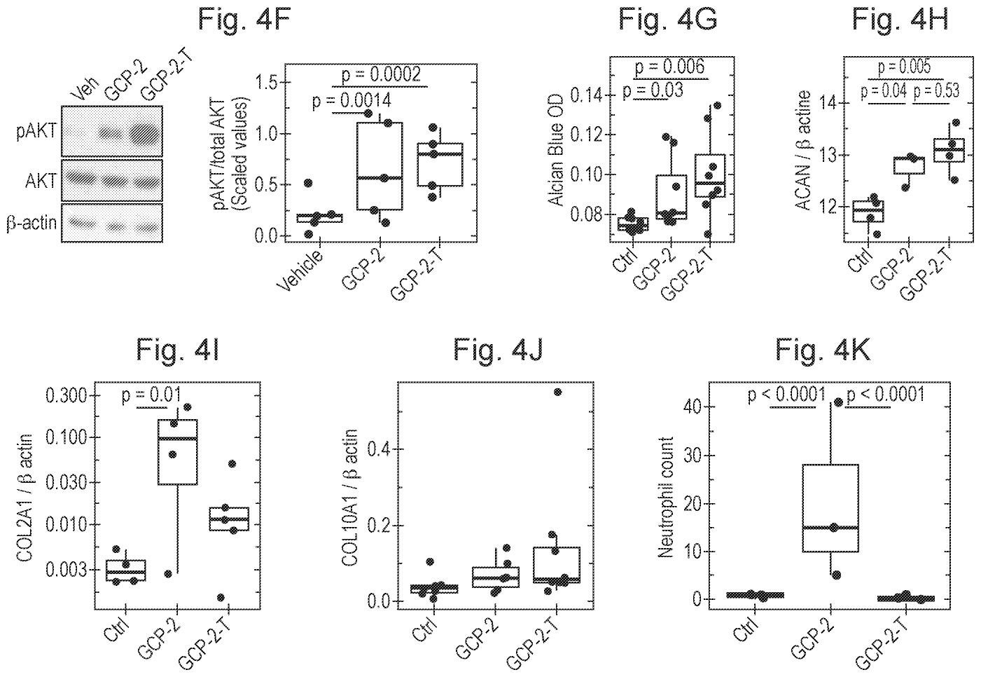

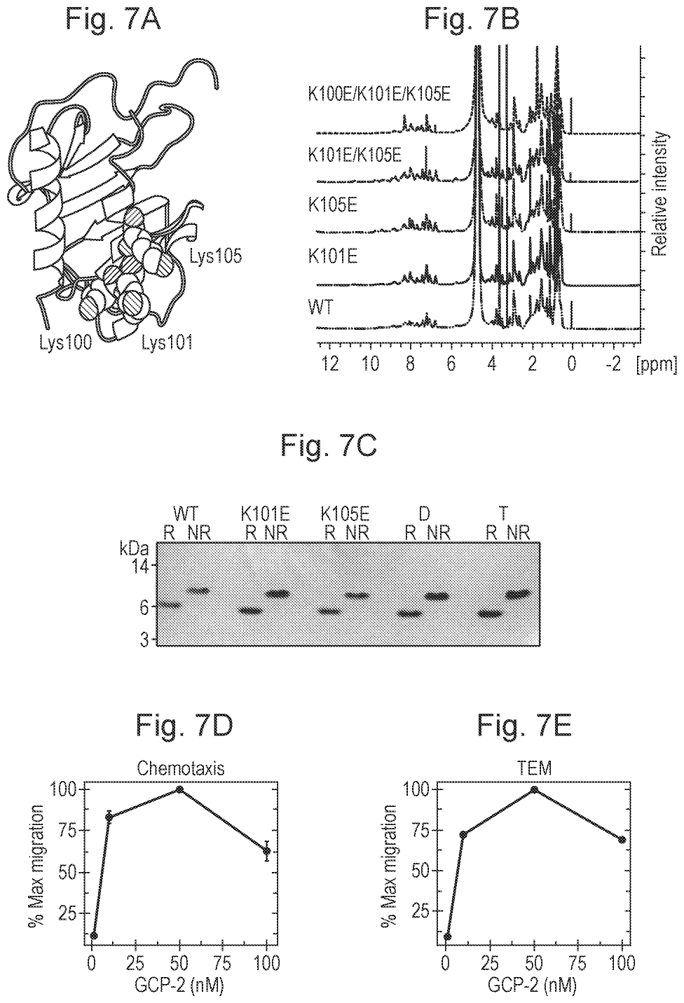

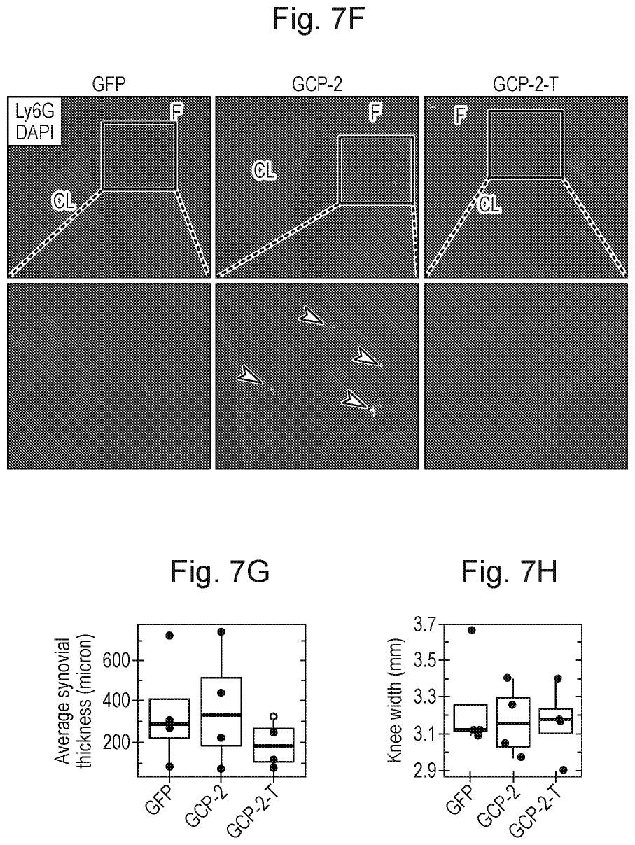

We hypothesized that disrupting the capacity of GCP-2 to bind GAGs on the endothelial surface might render GCP-2 unable to cause neutrophil chemotaxis while maintaining its capacity to activate its receptors on chondrocytes, and therefore retaining its chondrogenic activity. We predicted the position of the GAG-binding site based on a multiple sequence alignment of related chemokines (FIG. 4A) and identified a cluster of three highly conserved lysine residues (K100, K101 and K105) that could contribute to GAG binding (blue arrows in FIG. 4A). We generated two single site mutants, K101E and K105E, respectively, a double mutant (GCP-2-D) that incorporates both mutations and a triple mutant (GCP-2-T) that contains an additional K100E mutation. Importantly these mutations were not predicted to disrupt the tertiary structure of the protein as assessed by homology modeling of GCP-2 (FIG. 7A), which was confirmed by the essentially identical 1D NMR spectra (FIG. 7B) and migration patterns on SDS-PAGE under reduced and non-reduced conditions (FIG. 7C) for wild-type and mutant proteins; in the latter, the difference in apparent molecular weight of the reduced and non-reduced proteins are consistent with that reported for other chemokines.

The mutations resulted in a progressive decrease in binding to the GAG heparin, with the triple mutant showing the highest impairment and weakest binding (FIGS. 4B and 4C). In dose-response curves, wild-type (WT) GCP-2 induced optimal chemotaxis and optimal transendothelial migration in Boyden chambers at a concentration of 50 nM (FIGS. 7D and 7E). At the same concentration, both chemotaxis and endothelial migration were progressively affected by the mutations, with transendothelial migration reduced to a greater extent (FIGS. 4D and 4E).

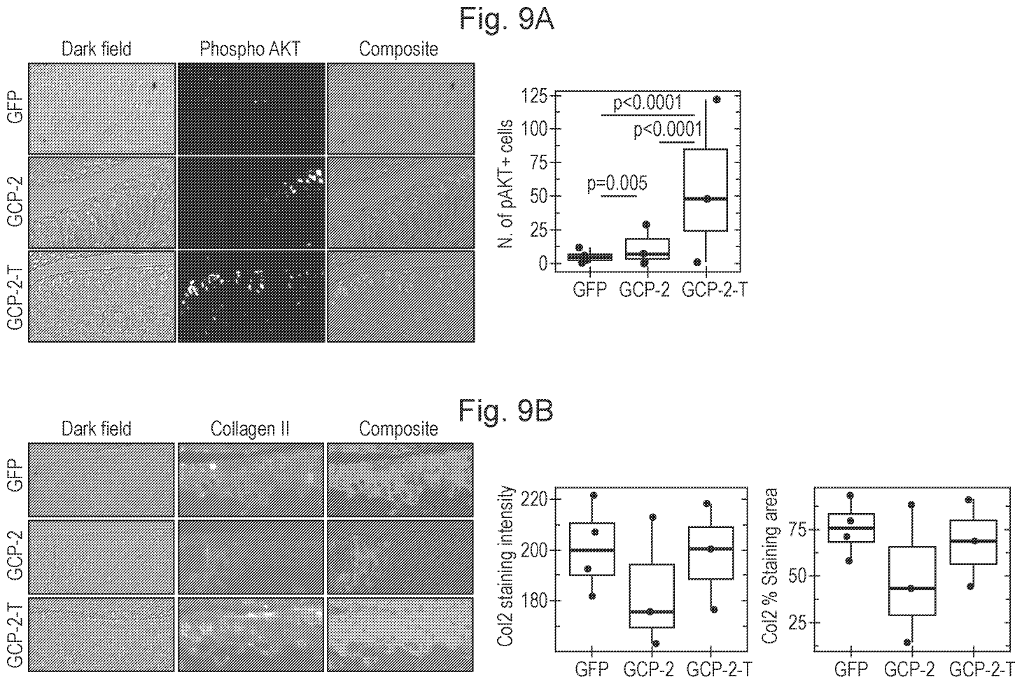

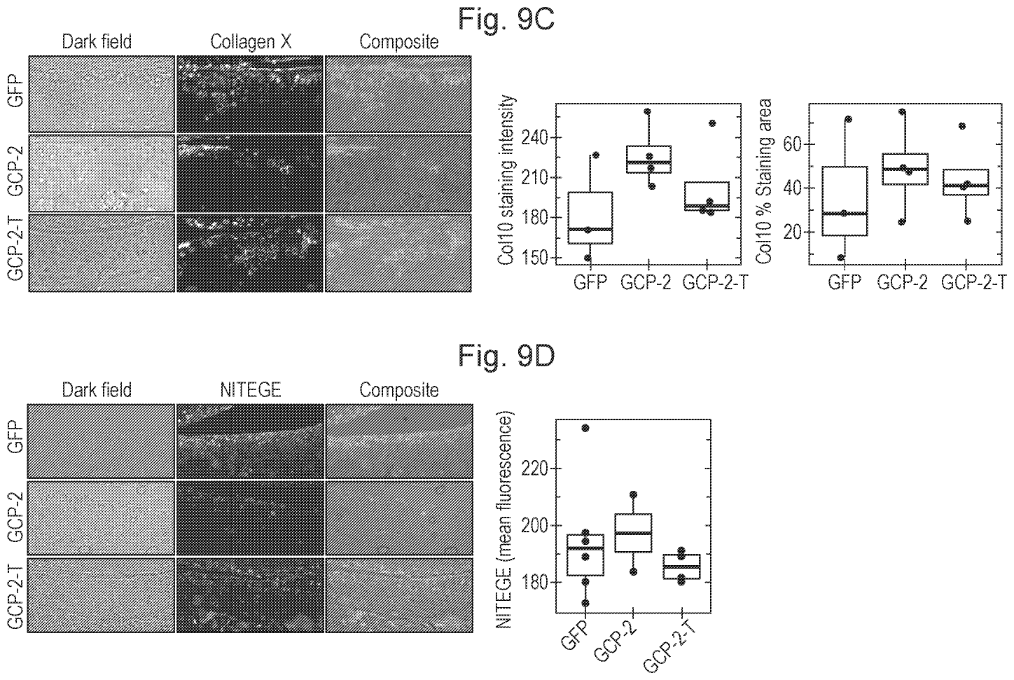

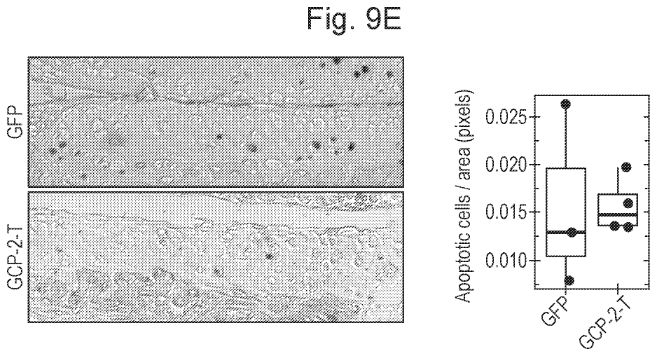

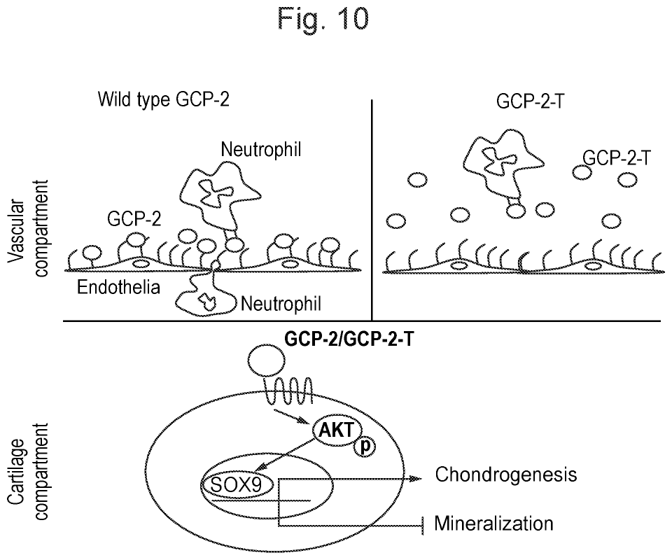

Despite the loss of its chemoattractive properties, the GCP-2-T was still able to induce AKT phosphorylation in chondrocytes (FIG. 4F), and promoted chondrogenesis, as assessed by Alcian blue staining (FIG. 4G) and aggrecan upregulation (FIG. 4H). Under these experimental conditions we could not detect statistically significant upregulation of COL2A1 by GCP-2-T (FIG. 4I). Although GCP-2 inhibited COL10A1 expression in osteogenic conditions (FIG. 3E), neither GCP-2 nor GCP-2-T affected baseline expression of COL10A1 (FIG. 4J). To confirm that GCP-2-T reduced chemoattractive activity in vivo, within the joint environment, we intra-articularly injected adenoviruses encoding wild type GCP-2, GCP-2-T or GFP as control. After 4 days, although both GCP-2 and GCP-2-T induced AKT phosphorylation (FIG. 9A), only GCP-2 induced accumulation of neutrophils within the joint space compared to GFP (FIG. 4K—quantification—and FIG. 7F for representative images). No difference between the treatments was detected in overall synovial thickness (FIG. 7G) and knee swelling as measured using a caliper (FIG. 7H). Taken together, these data show that mutations in the GAG-binding site of GCP-2 disrupted its capacity to induce chemotaxis and transendothelial migration of neutrophils in vitro and in vivo, but not the ability to activate downstream signaling in chondrocytes.

### Exogenous GCP-2-T Improves Pain and Structural Outcomes in Osteoarthritis

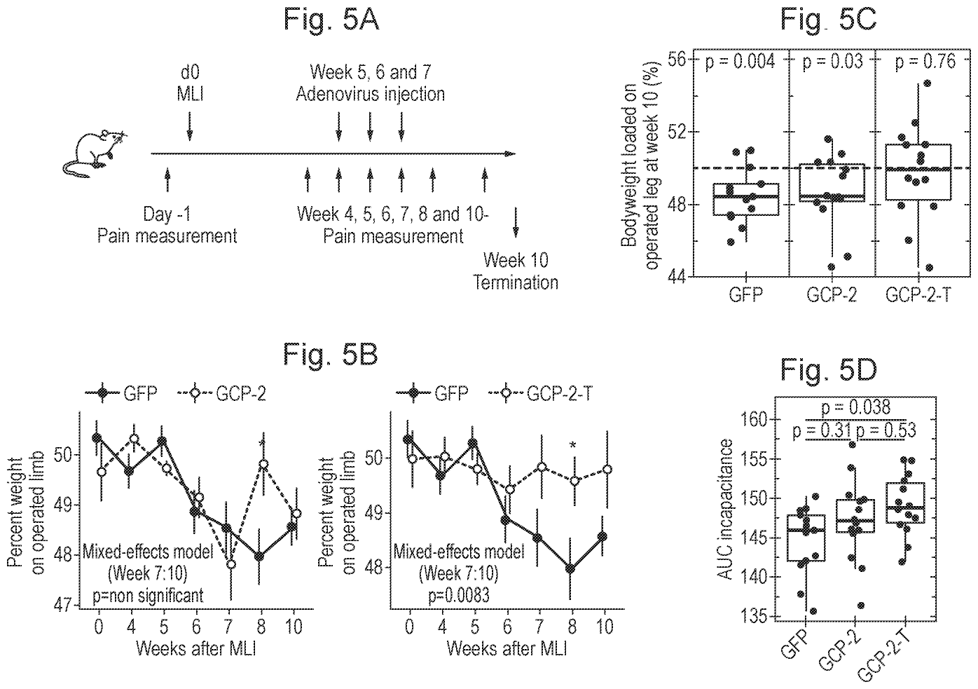

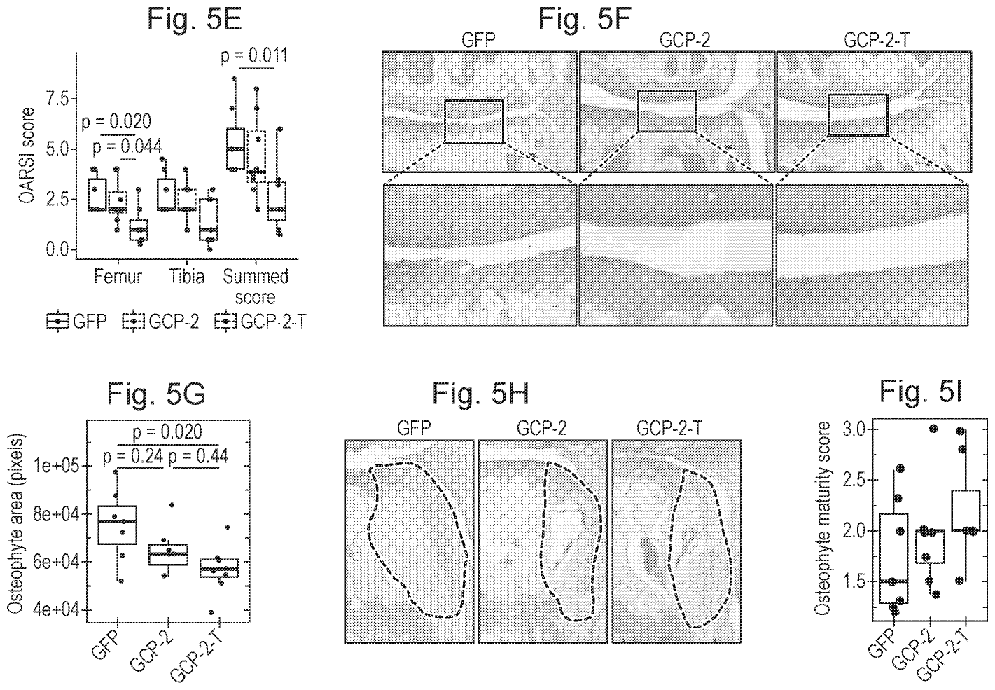

Having reduced the chemotactic function of GCP-2 by impairing GAG binding, we investigated whether administration of GCP-2-T in a therapeutic regime could improve the outcome of instability-induced osteoarthritis following menisco-ligament injury (MLI). Mice received three weekly intra-articular injections of adenoviruses encoding wild type murine GCP-2, GCP-2-T or GFP as control, starting five weeks after surgery, a time when cartilage lesions are well established (FIG. 5A). All mice were killed 10 weeks after surgery. As expected, the control group developed pain on weight bearing (incapacitance) as measured by the percentage of body weight loaded on the operated limb from week 6 (FIG. 5B-D). Mice treated with GCP-2-T showed no pain, whereas wild type GCP-2 did not significantly improve pain levels (FIG. 5B-D).

Reduced pain was accompanied by a reduced degree of structural damage in the medial compartment, with less cartilage loss in the GCP-2-T group, as assessed by OARSI score (FIGS. 5E and 5F), and reduced osteophyte size (FIG. 5G, 5H and 5I). As anticipated, subchondral bone density increased in the osteoarthritic knees compared to the sham operated knees (FIG. 9A), however no difference was observed between treatment groups (FIG. 9B). Reassuringly, no difference in synovial thickness was detected at this time point (FIG. 9C), suggesting no pro-inflammatory effect of GCP-2-T. No statistically significant difference in the expression of collagen type II, collagen type X, NITEGE neo-epitope and apoptosis were detected at this late time point (FIG. 9B-E).

### DISCUSSION

Osteoarthritis is a major cause of pain and disability for which there are currently no disease-modifying treatments available. We showed that by disrupting GAG binding, we could dissociate the anabolic function of GCP-2 from its capacity to induce chemotaxis of inflammatory cells. Intra-articular delivery of GAG binding-deficient mutant GCP-2-T in therapeutic regime improved pain and prevented cartilage loss in experimental murine osteoarthritis.

Boosting articular cartilage anabolism has long been advocated as a strategy for treatment of osteoarthritis. Transforming Growth Factor (TGF)-β and bone morphogenetic proteins (BMPs) are potent chondrogenic molecules, yet they have failed largely because they drive maturation of chondrocytes towards hypertrophy. Hypertrophic differentiation is a well-established driver of osteoarthritis progression. Therefore, over-activation of TGF-β signaling results in exacerbated osteoarthritis whereas suppression of TGF-β protects from osteoarthritis progression. Equally, BMP2 overexpression leads to ectopic bone formation.

Not only were the anabolic properties of GCP-2 not associated with hypertrophic differentiation, but—in fact—inhibited it. GCP-2 supported the “stable articular cartilage phenotype”, which was shown to be associated with a favorable outcome in cartilage repair interventions such as autologous chondrocyte implantation.

During embryonic development, GCP-2 is expressed in the portion of the cartilage forming the skeletal elements, which resists endochondral bone formation and will form the permanent articular cartilage. While this is outside the scope of this work, it is reasonable to speculate that the embryonic expression of GCP-2 in the prospective articular cartilage may contribute to its capacity to resist endochondral bone formation during development, whereas, in adult life, contributes to prevent mineralization and hypertrophic differentiation which are pathologic features in osteoarthritis.

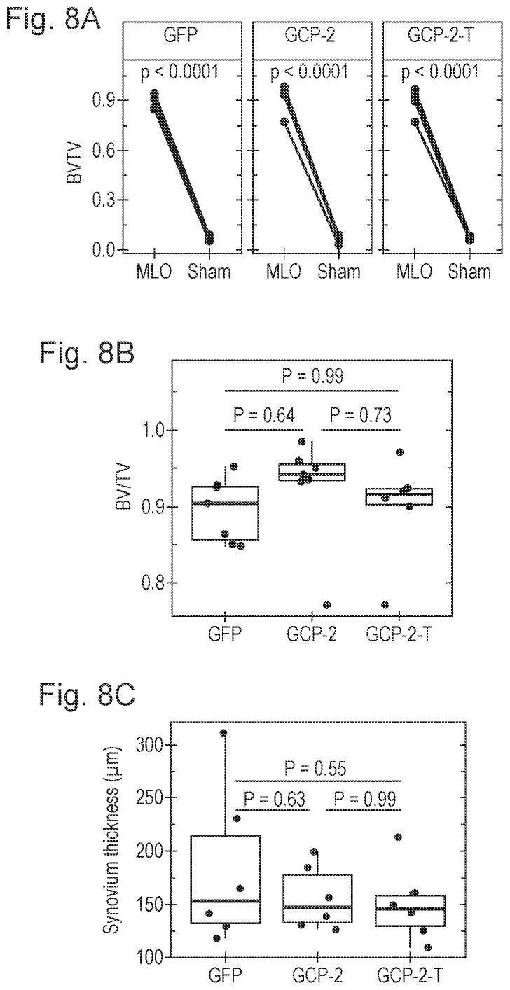

We dissociated the chemotactic properties of GCP-2 from its function as a chondrocyte differentiation factor by interfering with the binding to GAGs, which are present ubiquitously in the ECM (FIG. 4). Binding to GAGs is believed to be essential for the formation of a chemokine haptotactic gradient on the surface of blood vessel endothelium which activates neutrophil transendothelial migration. By disrupting the capacity of GCP-2 to adhere to the endothelial surface we disrupted its capacity to attract neutrophils, while retaining its capacity to activate chemokine receptors on chondrocytes. Additionally, the fact that GCP-2-T was more efficient than wild-type GCP-2 in inducing AKT phosphorylation in vivo (FIG. 9A) but not in vitro (FIG. 8F) suggests that disruption of GCP-2 GAG binding within the cartilage ECM might have made it more bioavailable for chondrocytes and therefore contributed to its efficacy. Within cartilage, ECM binding sequesters growth factors including FGF2, BMPs, and TGF-β/CTGF. Loss of GAGs or loss of the interaction between the GAGs and growth factors, resulted in growth factor release and in activation of the respective signaling pathways.

In our osteoarthritis model, we do not have an arm in which mice were killed before starting treatment, and therefore it is not possible to discriminate whether GCP-2-T caused cartilage regeneration or merely decreased breakdown. However, the fact that GCP-2 induced extracellular matrix accumulation in human cartilage organoids implanted in nude mice (FIG. 2F) suggests an anabolic function.

From a translational point of view, our data suggest that GCP-2-T can represent a first-in-kind disease-modifying osteoarthritis drug. As osteoarthritis is a leading cause of permanent disability for which we do not yet have an effective pharmacological treatment, the use of GCP-2-T may represent a game-changer in a high-priority area of modern medicine.

Compared to other molecules which have shown some efficacy in osteoarthritis, GCP-2-T has the highly desirable feature of coupling disease modification (cartilage integrity) with rapid pain relief. This is important because cartilage integrity without pain relief is not of benefit to patients: for instance, FGF18 was effective in terms of cartilage integrity but failed to improve pain even after 5 year follow up. Conversely, pain relief without cartilage protection is also unhelpful: tanezumab induced pain relief but resulted in dose-dependent accelerated OA in some patients, likely due to the increased use of the joint in the absence of chondroprotection.

It is still unclear how GCP-2-T mediates pain relief. It is possible that it may control local inflammation by competing with other inflammatory chemokines for the binding to CXCR2, however, at this stage, we cannot exclude that it may directly signal to local nociceptors within the joints. These studies are currently ongoing.

One limitation of this study is that we used adenoviral overexpression rather than a recombinant molecule to provide proof of concept of efficacy in osteoarthritis. The generation and validation of a recombinant GCP-2-T with a pharmacokinetic profile suitable for not-too-frequent intra-articular injections is currently being pursued.

For its anabolic and possibly anti-inflammatory properties, we anticipate that GCP-2-T may represent a novel therapeutic tool not only in osteoarthritis, but perhaps also in inflammatory arthritis where cartilage damage is the final disabling outcome.

### Materials and Methods

### Ethics

All animal procedures were subjected to, and complied with local ethical approval and Home Office Licensing. Mouse experiments were regulated by Procedure Project Licence (PPL) nos. 70/7986 and 60/4528. Human samples were approved and obtained under the East London and the City Research Ethics commitee3 (Ethics approval Rec N. 07/Q0605/29) and by the KULeuven Ethics Committee (OG032), application ML3356 and ML3359, by decision of 8 Sep. 2004. The experiments conformed to the principles set out in the WMA Declaration of Helsinki and the Department of Health and Human Services Belmont Report. Adult human articular cartilage was obtained from patients undergoing joint replacement for knee OA after obtaining informed consent.

### Micromass Culture and Alcian Blue Staining

Primary human articular chondrocyte isolation and expansion was performed as previously described. Cells were cultured in micromass, and stained with alcian blue as previously described. These were used for densitometry quantification which was measured using ImageJ, as previously described.

For gain of function experiments, GCP-2 or vehicle control was added at a final concentration of 100 ng/ml and the micromasses were cultured for additional 3 days unless stated otherwise. For loss of function experiments, densitometry quantification of alcian blue was done for: HAC micromasses stimulated with 12.5 μg/ml GCP-2 blocking antibody (Bio-Techne MAB333) or IgG control (MAB002); C3H10T½ cells (ATCC CCL-226) and C28/I2 cells (a gift from M. Goldring (HSSResearch Institute, Hospital for Special Surgery, New York, New York) were transfected with 25 nM of GCP-2 siRNA (Thermofisher Silencer™ Select Pre-Designed siRNA Catalog #4392420) for 24 h and cultured in micromass. siRNA sequences: sense CUAUUGUAUUUCUAUCAUATT; antisense UAUGAUAGAAAUACAAUAGTT.

### Hypertrophic and Osteogenic Differentiation

Hypertrophy was induced in HACs as previously described. Osteogenic differentiation was induced in C3H10T½ cells as previously described. After 3 or 4 weeks (hypertophic or osteogenic differentiation, respectively) cells were harvested for real-time PCR gene expression analysis or fixed and stained for alkaline phosphatase (Vector Red) or alizarin red, as previously described.

### Total RNA Extraction and Real-Time PCR

RNA extraction was performed using Trizol® (Invitrogen) according to the manufacturer's instruction. Reverse transcription and real-time PCR were performed as previously described. Primers are listed in Table 2.

### Western Blot Analysis and Immunostaining

Western blots were carried out as described previously. Membranes were blocked and antibodies diluted in 1% (w/v) BSA+0.01% (v/v) Tween 20 in TBS. Primary antibodies are listed in Table 3. Immunofluorescence staining was carried out as previously described. Antigen retrieval methods for each staining are listed in Table 3. For Ly6G immunostaining only Tyramide signal amplification (TSA) was used using the ABC kit (Vector Laboratories).

### Generation of Recombinant GCP-2 and Mutants

The cDNA of human WT GCP-2 (accession number NM_002993 bases 185-415) or the GCP-2 mutants were cloned into the pRK172 T7 expression vector (by GenScript, USA); an N-terminal 6×His tag was included followed by a factor Xa cleavage site in order to allow production of the native N-terminus. K101E and K105E single mutants and a double mutant (GCP-2-D) incorporating both mutations, and a triple mutant (GCP-2-T) containing an additional mutation K100E were generated.

WT and mutant plasmids were transformed into SHuffle T7 Express lysY Competent E. coli cells (NEB, USA) and recombinant protein expressed at 16° C. and purified using standard methodology. Briefly, following IPTG induction cells were harvested and resuspended in IMAC buffer (20 mM sodium phosphate, 500 mM NaCl, pH 7.4) supplemented with protease inhibitor cocktail (Sigma-Aldrich) and lysed by sonication. Supernatants were collected by centrifugation and loaded onto a 1-ml HisTrap HP column (GE Healthcare) and protein eluted with a 0-500 mM gradient of imidazole in IMAC buffer; GCP-2 proteins were pooled and dialysed against 20 mM Tris-HCl pH7.4, 100 mM NaCl. CaCl₂ was added (2 mM) and the 6×His tag cleaved by factor Xa (overnight) according to manufacturer's instructions (Millipore, USA) and removed by retention on the HisTrap HP column (run in 20 mM Tris-HCl pH 7.0, 100 mM NaCl). The GCP-2 proteins were loaded onto a 1 ml HiTrap Heparin HP column equilibrated with 20 mM Tris-HCl pH 7.0, 100 mM NaCl and eluted with a gradient 0.1M-1.5M NaCl in 20 mM Tris-HCl pH 7.0. Fractions were pooled and the protein was snap-frozen and stored at −20° C. The WT and mutant GCP-2 proteins (0.3 mg/ml) were analysed by 1-dimensional ¹H nuclear magnetic resonance (NMR) spectroscopy on a 500 MHz Bruker NMR spectrometer in 20 mM sodium phosphate, 100 mM NaCl, pH 7.0, 10% (v/v) D₂O. This confirmed that the mutations did not have any deleterious effects on the GCP-2 structure, and all mutants had a WT fold. For SDS-PAGE analysis under reducing conditions, protein samples (1 μg) were incubated in 100 mM Tris-HCl, pH 8.0, 20 mM DTT, 4 M Urea and 1% (w/v) SDS for 30 min at 37° C. and then alkylated by addition of iodoacetamide to 40 mM and incubated at 37° C. for 5 min, or under non-reducing conditions, samples were incubated in 100 mM Tris-HCl, pH 8.0, 40 mM iodoacetamide, 4 M Urea, and 1% (w/v) SDS for 5 min at 37° C. The protein samples were run on a 4%-12% Bis-Tris SDS PAGE gel (Invitrogen, UK) and then stained with Ready Blue Protein Gel Stain (Merck, UK).

### Heparin-Binding Assays

The heparin-binding activities of WT and mutant GCP-2 were compared using a microtiter plate assay, carried out as described previously.

The relative heparin binding affinities of GCP-2 WT and mutants were also assessed by affinity chromatography on a HiTrap Heparin HP 1 ml column (GE Healthcare) as described previously. Briefly, GCP-2 or mutants (50 μg) were loaded onto the column equilibrated in PBS, pH 7.3 and the proteins were eluted using a linear gradient of 0-2M NaCl in the same buffer and absorbance monitored at 215 nm.

### Chemotaxis and Trans-Endothelial Migration Assay

The chemotaxis and transendothelial migration assays were performed as previously. Chemotaxis was assessed using 6.5 mm Transwell permeable supports in 24-well plates (5 μm pore polycarbonate membrane; Corning) with CXCR2-expressing 300-19 pre-B cells. For transendothelial migration assays, 5×10⁴ EA.hy 926 cells were seeded on 6.5 mm Transwell inserts. Monolayer formation was confirmed by microscopy. 300-19 cells were used as above.

To assess in vivo neutrophil migration, 8 week old female C57BL/6 mice (purchased from Charles River UK) received intra-articular injections of 10 μl 10⁹ PFU of adenoviral constructs and culled 48h later. Mice were maintained in standard housing in groups of six and fed ad libitum, the investigator was blinded to the treatment being injected. The infiltration of neutrophils into the knee joint was evaluated using immunofluorescence staining for Ly6G marker. The number of neutrophils was counted in intercondylar area.

### In Vivo Ectopic Cartilage Formation Assay

The ectopic cartilage (EC) formation assay was modified from previously described protocols. For each injection, 1×10⁶ chondrocytes were mixed with 1×10⁵ COS7 cells (a gift from M. Ferns (UC Davis Healthsystem, USA)) transfected with GFP or GCP-2 plasmids and growth arrested with mitomycin C, as described previously. The cell mixture was resuspended in 100 μl rat type I collagen (Corning) at pH 7.2-7.6 and injected subcutaneously in the back of 3-week old female CD1-Foxn1^{nu} mice. Mice were maintained in standard housing in groups of six and fed ad libitum. After two weeks mice were sacrificed, and cartilage organoids were retrieved. Harvested organoids were analyzed as previously described.

### Adenoviruses

The cDNA encoding the mature region of mouse GCP2 (genebank NM_009141.3) or the triple mutant version or EGFP were cloned downstream of a Igk signal peptide and followed by a stop codon into Ad5 adenovirus backbone. All adenoviruses were constructed and packaged at Vectorbuilder.

### Experimental Osteoarthritis in Mice

Osteoarthritis was induced in 10-week-old male C57BL/6 mice (purchased from Charles River UK) by meniscus-ligament injury (MLI) as previously described −45 animals in total. Contralateral knees were sham-operated. Within each cage, animals were block-randomized to receive three weekly intra articular injections of 10 μl 10⁹ PFU of adenovirus encoding mouse GFP or GCP-2 or GCP-2T starting from week 5 (FIG. 9A) within the operated knee. n=15 animals were used per treatment group, where sample size was based on power calculation of a previous study using this model. All mice were maintained in standard housing in groups of six and fed ad libitum. Investigators performing the surgery, the injection, the analysis of pain and the histological scoring were blinded to the treatment. Two mice in the GCP-2-T group were excluded from downstream analysis due to an accidental breach of protocol.

### Incapacitance

Incapacitance was measured throughout the study using a Linton incapacitance meter. Measurements were taken over 3 seconds, and 10 readings per mouse were taken at each time-point, and mean incapacitance was calculated. The area under the curve for each mouse was calculated from weeks 6 to 10 after surgery.

### Osteoarthritis Scoring

OARSI scoring was performed as previously described on Safranin O stained sections. Sections within the load-bearing area (200 μm from the section through the middle of the tibial plateau) were collected at an 80 μm interval and stained with 0.1% Safranin-O pH 5. Sections with cutting or staining artefacts were discarded. All images were taken using the same settings on NanoZoomer S60 slide scanner microscope and osteoarthritis severity in the medial compartment was assessed using the OARSI score. Sections were scored independently by two investigators (FD and SC) who were blind to the treatment and the individual scores were averaged. The area and density of osteophytes were measured in a similar way, by manually selecting the region of interest using ImageJ. The size and morphology of osteophytes were scored using the scoring system from. Knees with <3 optimal sections for assessment of osteoarthritis severity were excluded from analysis.

### Micro-CT

Micro-CT analyses were performed using a Skyscan 1176 microtomograph (Bruker) at 9 μm resolution through a 0.5 mm aluminium filter. Images were analysed with Image J using Bone J software for ratio of bone volume to total volume (BV/TV) in regions of interest (ROI) of the medial tibial epiphyseal bone fraction.

### Image Treatment

Within each experiment, images were obtained using the same setting and without autogain. Where needed, images have been edited for best rendering (contrast, brightness and hue) using the same parameters within each experiment so not to alter the differences between samples and experimental groups.

### Statistical Analysis

Data shown in figures represent mean values±standard error. Parametric data were compared with student's t-test or analysis of variance (ANOVA) followed by Tukey's honest significant difference (HSD) post hoc test for multiple comparisons. Data transformation was applied to satisfy the assumption of the t-test and ANOVA, when applicable as described. When the assumptions for ANOVA were not fulfilled, means were compared by fitting generalized linear models followed by pairwise comparison of the estimated marginal means in the treatment groups. Tests were two-sided and p values<0.05 were considered significant. Individual tests used and specific p-values are indicated in figure legends. Raw data and R scripts to reproduce statistical analyses and graphs are downloadable as supplementary materials.

TABLE 2
List of primers

| Primer Name | Primer Sequence |
|---|---|
| Human and Mouse B | TGACGGGGTCACCCACACTGTGCC |
| Actin Forward | CATCTA |
| Human and Mouse B | CTAGAAGCATTTGCGGTGGACGAT |
| Actin Reverse | GGAGGG |
| Mouse Aggrecan Forward | GTTGTCATCAGCACCAGCATC |
| Mouse Aggrecan Reverse | ACCACACAGTCCTCTCCAGC |
| Human RUNX2 Forward | CGGACATACCGAGGGACATG |
| Human RUNX2 Reverse | CCAACCCACGAATGCACTATC |
| Human Col10A1 Forward | AATCCCTGGACCGGCTGGAATTTC |
| Human Col10A1 Reverse | TTGATGCCTGGCTGTCCTGGAACC |
| Human Col1A1 Forward | CGTGGTGACAAGGGTGAGAC |
| Human Col1A1 Reverse | TAGGTGATGTTCTGGGAGGC |

TABLE 3
Antibodies and working concentrations

| Antibody | Dilution | Supplier | Antigen retrieval |
|---|---|---|---|
| β Actin | 1:10000 | Abcam | |
| Akt | 1:1000 | CST | |
| pAkt | 1:1000 | CST | |
| Collagen II | 1:500 | Millipore | |
| GFP | 1:100 | CST | |
| GCP-2 | 1:100 | R&D Systems | |
| GCP-2 | 1:100 | Byorbit | |
| Lix | 1:100 | R&D Systems | |
| His | 1:400 | CST | |
| Collagen II | 1:500 | Millipore | Pepsin digestion |
| Collagen X | 1:100 | Ebioscience | Pepsin digestion |
| | | | Citrate-EDTA |
| MMP-13 | 1:100 | Abcam | buffer (pH 6.2) |
| GCP-2 | 1:200 | Byorbit | Pepsin digestion |
| NITEGE | 1:50 | MD Bioproducts | Proteinase K |
| pAkt | 1:200 | CST | Pepsin digestion |
| Ly6G | 1:100 | Biolegend | Citrate Buffer |
| TUNEL assay | | Sigma Aldrich | Proteinase K |

SEQUENCES

SEQ ID NO: 1
MSLPSSRAARVPGPSGSLCALLALLLLLTPPGPLASAGPVSAVLTELRC
TCLRVTLRVNPKTIGKLQVFPAGPQCSKVEVVASLKNGKQVCLDPEAPF
LKKVIQKILDSGNKKN
SEQ ID NO: 2
TGACGGGGTCACCCACACTGTGCCCATCTA
SEQ ID NO: 3
CTAGAAGCATTTGCGGTGGACGATGGAGGG
SEQ ID NO: 4
GTTGTCATCAGCACCAGCATC
SEQ ID NO: 5
ACCACACAGTCCTCTCCAGC
SEQ ID NO: 6
CGGACATACCGAGGGACATG
SEQ ID NO: 7
CCAACCCACGAATGCACTATC
SEQ ID NO: 8
AATCCCTGGACCGGCTGGAATTTC
SEQ ID NO: 9
TTGATGCCTGGCTGTCCTGGAACC
SEQ ID NO: 10
CGTGGTGACAAGGGTGAGAC
SEQ ID NO: 11
TAGGTGATGTTCTGGGAGGC

### Exemplary Molecules of the Invention

| SEQ ID | Sequence of exemplary molecule | Description |
|---|---|---|
| NO: 12 | MSLPSSRAAR VPGPSGSLCA LLALLLLLTP PGPLASAGPV | GCP-2 K101E |
| | SAVLTELRCT CLRVTLRVNP KTIGKLQVFP AGPQCSKVEV | (single mutant) |
| | VASLKNGKQV CLDPEAPFLK EVIQKILDSG NKKN | |
| NO: 13 | MSLPSSRAAR VPGPSGSLCA LLALLLLLTP PGPLASAGPV | GCP-2 K105E |
| | SAVLTELRCT CLRVTLRVNP KTIGKLQVFP AGPQCSKVEV | (single mutant) |
| | VASLKNGKQV CLDPEAPFLK KVIQEILDSG NKKN | |
| NO: 14 | MSLPSSRAAR VPGPSGSLCA LLALLLLLTP PGPLASAGPV | GCP-2-D |
| | SAVLTELRCT CLRVTLRVNP KTIGKLQVFP AGPQCSKVEV | (double mutant K101E, K105E) |
| | VASLKNGKQV CLDPEAPFLK EVIQEILDSG NKKN | |
| NO: 15 | MSLPSSRAAR VPGPSGSLCA LLALLLLLTP PGPLASAGPV | GCP-2-T |
| | SAVLTELRCT CLRVTLRVNP KTIGKLOVFP AGPQCSKVEV | (triple mutant |
| | VASLKNGKOV CLDPEAPFLE EVIQEILDSG NKKN | K100E, K101E, K105E) |
| NO: 16 | MSLPSSRAAR VPGPSGSLCA LLALLLLLTP PGPLASAGPV | GCP-2-D2 |
| | SAVLTELRCT CLRVTLRVNP KTIGKLQVFP AGPQCSKVEV | (double mutant K100E, K101E) |
| | VASLKNGKQV CLDPEAPFLE EVIQKILDSG NKKN | |
| NO: 17 | MSLPSSRAAR VPGPSGSLCA LLALLLLLTP PGPLASAGPV | GCP-2-D3 |
| | SAVLTELRCT CLRVTLRVNP KTIGKLOVFP AGPQCSKVEV | (double mutant K100E, K105E) |
| | VASLKNGKQV CLDPEAPFLE KVIQEILDSG NKKN | |
| NO: 18 | MSLPSSRAAR VPGPSGSLCA LLALLLLLTP PGPLASAGPV | GCP-2 K100E |
| | SAVLTELRCT CLRVTLRVNP KTIGKLOVFP AGPQCSKVEV | (single mutant) |
| | VASLKNGKQV CLDPEAPFLE KVIQKILDSG NKKN | |

1. A polypeptide ligand of chemokine receptor CXCR1 and/or CXCR2, which:
a. has an ability to activate CXCR1 and/or CXCR2; and
b. has a reduced ability to bind to glycosaminoglycans (GAGs) as compared to GCP2 of SEQ ID NO: 1;
wherein said polypeptide comprises or consists of the sequence of SEQ ID NO: 1 in which at least one positively charged amino acid has been substituted for a negatively charged or neutral amino acid, or in which at least one positively charged amino acid has been chemically modified to neutralise the positive charge.

2. A polypeptide ligand according to claim 1 which has a reduced ability to attract inflammatory cells as compared to GCP2 of SEQ ID NO: 1.

3. A polypeptide ligand according to claim 1, wherein said at least one positively charged amino acid is:
a. not within a CXC motif of GCP2; and/or
b not within an ELR motif of GCP2.

4. A polypeptide ligand according to claim 1, wherein said at least one positively charged amino acid is lysine or arginine.

5. A polypeptide ligand according to claim 1, wherein said negatively charged or neutral amino acid is glycine, alanine, valine, leucine, isoleucine, histidine, methionine, phenylalanine, tryptophan, proline, serine, threonine, cysteine, tyrosine, asparagine, glutamine, aspartic acid or glutamic acid, and/or wherein said chemical modification is succinylation, PEGylation or treatment with DEPC or p-Hydroxyphenylglyoxal.

6. A polypeptide ligand according to claim 1,
wherein said positively charged amino acid is at:
(i) any one of K100, K101 or K105;
(ii) any two of K100, K101 or K105 (that is K100 and K101; K100 and K105; or K101 and K105); or
(iii) all three of K100 and K101 and K105.

7. A polypeptide ligand according to claim 6, wherein at least one of the substitutions in (i), (ii) or (iii) is with E.

8. A polypeptide according to claim 7, which comprises or consists of the amino acid sequence of any one of SEQ ID NOs: 12 to 18.

9. A polynucleotide which encodes a polypeptide ligand according to claim 1.

10. A composition comprising the polypeptide ligand according to claim 1, which comprises at least one pharmaceutically acceptable diluent, carrier, preservative or excipient.

11. A method of treating or preventing a disease or condition in a subject, the method comprising administering to the subject the polypeptide ligand according to claim 1.

12. A method of treating or preventing a disease condition in a subject according to claim 11, wherein the disease or condition is characterised by degeneration of cartilage, damage to cartilage, or a need for cartilage regeneration.

13. A method according to claim 11, wherein the disease is characterised by pain or stiffness, optionally wherein the pain is nociceptive and/or neuropathic pain.

14. A method according to claim 11, wherein the disease is further characterised by hypertrophic differentiation, chondrocyte hypertrophy, calcification, and/or bone formation.

15. A method according to claim 11, wherein the disease is osteoarthritis.
